(19) United States
(12) Patent Application Publication
(10) Pub. No.: US 2026/0201450 A1
(43) Pub. Date: Jul. 16, 2026

(54) CRISPR-BASED SINGLE NUCLEOTIDE POLYMORPHISM DETECTION VIA PROGRAMMED MISMATCHES

(71) Applicant: BOARD OF REGENTS, THE UNIVERSITY OF TEXAS SYSTEM, Austin, TX (US)

(72) Inventors: Ilya J. FINKELSTEIN, Austin, TX (US); Hung-Che KUO, Austin, TX (US); Chia-Wei CHOU, Austin, TX (US)

(21) Appl. No.: 19/135,733

(22) PCT Filed: Dec. 22, 2023
(86) PCT No.: PCT/US2023/085762

§ 371 Date: Jun. 4, 2025

Related U.S. Application Data
(60) Provisional application No. 63/434,978, filed on Dec. 23, 2022.

(51) Int. Cl. C12Q 1/6827

(52) U.S. Cl. CPC C12Q 1/6827

(57) ABSTRACT

The present invention relates to a CRISPR-based method of detecting a target nucleic acid, wherein the detection is via programmed mismatches between crRNA and the target nucleic acid. Also disclosed herein is a CRISPR-based platform for detecting whether a sample comprises a target nucleic acid. Said platform can be utilized for diagnosing or determine if a disease or an infection is present in a subject.

### I. CROSS-REFERENCE TO RELATED APPLICATIONS

This application claims benefit of U.S. Provisional Application No. 63/434,978, filed Dec. 23, 2022, incorporated herein by reference in its entirety.

### II. BACKGROUND

Single nucleotide polymorphisms (SNPs) have been shown to be involved in disease susceptibility, disease pathogenesis, and drug efficacy. For instance, the omicron variant of SARS-CoV-2 has several SNPs that were known to increase virus transmission. Detection of pathogen variants or pathogenic gene variants is critical for optimal personalized therapy. Common methods for SNP detection are PCR-based methods or whole genome sequencing. However, these methods usually require lab equipment and/or time consuming.

Clustered regularly interspaced short palindromic repeats (CRISPR)-based diagnostics can allow for rapid nucleic acid discrimination. CRISPR systems are a fundamental part of a microbial adaptive immune system that recognizes foreign nucleic acids on the basis of their sequence to subsequently eliminate them by means of endonuclease activity associated with the CRISPR-associated (Cas) enzyme. Although there are diverse CRISPR-Cas systems among the different species of archaea and bacteria, these systems are connected by their dependence on CRISPR RNA (crRNA), which guides Cas proteins to recognize and cleave nucleic acid targets. The crRNA can be programmed towards a specific DNA or RNA region of interest through hybridization to a complementary sequence, which in some systems is restricted to the proximity of a protospacer adjacent motif (PAM) or protospacer flanking sequence. CRISPR-Cas systems have been repurposed for a variety of applications, including the targeted editing of genomes, epigenomes and transcriptomes, the bioimaging of nucleic acids, the recording of cellular events and the detection of nucleic acids. However, none of these systems are able to (Kaminski. M. M., Abudayyeh, O. O., Gootenberg, J. S. et al. CRISPR-based diagnostics. Nat Biomed Eng 5, 643-656 (2021). doi.org/10.1038/s41551-021-00760-7).

What are needed are methods and systems for discriminating between target nucleic acids and a variant thereof in given regions of target nucleic acid.

### III. SUMMARY

Disclosed herein are CRISPR-based diagnostics take advantage of the collateral activity of Cas12/Cas13 effectors and can provide an accurate point-of-care detection. Current CRISPR-based diagnostics are focusing on detecting if a specific DNA/RNA is presented in the sample. By positioning a mismatch between the crRNA and the target RNA molecule, the study can control the signal of Cas13-based detection. This can be exploited to differentiate SNPs in CRISPR diagnostics applications. The instant disclosure shows mismatch positions between crRNA and target RNA in Cas13d that diminishes the cleavage activity of Cas13d. With programmed mismatches on specific positions, SNPs can be specifically identified by the difference of signal in Cas13d diagnostics.

Accordingly, disclosed herein is a method of detecting whether a sample comprises a target nucleic acid or a variant of the target nucleic acid, the method comprising: exposing the target nucleic acid to a CRISPR/Cas probe system; allowing the sgRNA to interact with the target nucleic acid or variant thereof in the presence of the Cas molecule and the nucleic acid probe, so that, upon hybridization of the crRNA with the target or variant thereof, the Cas molecule interacts with tracrRNA, and further wherein the Cas molecule cleaves the nucleic acid probe most efficiently when the crRNA is 100% complementary to the target nucleic acid; and measuring signal from the nucleic acid probe, wherein one signal indicates that the target nucleic acid is 100% complementary to the crRNA, and wherein another signal indicates that a variant of the target nucleic acid is present. In some examples, the system of any preceding aspect comprises: a single guide RNA (sgRNA) molecule, wherein said sgRNA comprises CRISPR RNA (crRNA) and tracrRNA, wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target; a Cas molecule, wherein said Cas molecule cleaves nucleic acid when the crRNA is 100% complementary to the target nucleic acid with the given region, but does not cleave nucleic acid, or cleaves nucleic acid less efficiently, when the crRNA is hybridized with a variant which comprises one or two mismatches in the given region; and a nucleic acid probe. wherein said nucleic acid probe is cleavable by the Cas molecule, and further wherein the probe gives a different signal when cleaved compared to when it is not cleaved.

In some embodiments, the sample is from a subject. In some embodiments, the variant of the target nucleic acid is a single nucleotide polymorphism (SNP). In some embodiments, the Cas molecule has non-specific cleavage activity. In some embodiments, the Cas molecule lacks protospacer flanking sequence recognition.

In some embodiments, the Cas molecule is Cas13. In some embodiments, the Cas molecule is Cas13d.

In some embodiments, the given region of step a) is at a proximal end in relation to the tracrRNA. In some embodiments, the given region is 1-15 nucleotides long or 1-11 nucleotides long. In some embodiments, the given region is a distal spacer region.

In some embodiments, the target and/or variant thereof comprises secondary structure, wherein said secondary structure affects crRNA hybridization kinetics.

In some embodiments, the probe comprises a fluorophore and quencher molecule. In some embodiments, the probe comprises a metal nanoparticle.

In some embodiments, in step (c), the presence of target nucleic acid causes a stronger cleavage reaction by Cas, and therefore a more intense signal, compared to a variant of the target nucleic acid.

In some embodiments, the method of any preceding aspect is high throughput. In some embodiments, the method of any preceding aspect is quantitative. In some embodiments, RNA chip-hybridized affinity-mapping platform (RNA-CHAMP) is used to carry out the method. In some embodiments, the method is done in multiplex so that more than one variant of a single target nucleic acid can be detected simultaneously. In some embodiments, said means of measuring further comprises a readout of a signal from the probe. In some embodiments, said readout of signal from the probe comprises ELISA. In some embodiments, said readout is a point of care diagnostic. In some embodiments, said point of care diagnostic is a handheld test. In some embodiments, said means of measuring comprises a computer system, wherein said computer system interprets the signal. In some embodiments, said computer system displays results.

Also disclosed herein is a platform for detecting whether a sample comprises a target nucleic acid or a variant of the target nucleic acid, wherein the platform comprises: a CRISPR/Cas detection system and a detection means, whereby said detection means indicates whether a target nucleic acid or variant thereof is present. In some examples, said CRISPR/Cas detection system comprises: a single guide RNA (sgRNA) molecule, wherein said sgRNA comprises CRISPR RNA (crRNA) and tracrRNA. wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target; a Cas molecule, wherein said Cas molecule cleaves nucleic acid when the crRNA is 100% complementary to the target nucleic acid with the given region, but does not cleave nucleic acid, or cleaves nucleic acid less efficiently, when the crRNA is hybridized with a variant which comprises one or two mismatches in the given region; and a nucleic acid probe, wherein said nucleic acid probe is cleavable by the Cas molecule, and further wherein the probe gives a different signal when cleaved compared to when it is not cleaved.

Also disclosed herein is a method for optimizing discrimination between a target nucleic acid and at least one variant of the target nucleic acid during detection, the method comprising: determining a target nucleic acid and a variant of the target nucleic acid for detection, wherein discrimination between the target and the variant is desired; designing an sgRNA molecule, wherein said sgRNA comprises crRNA and tracrRNA, wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target; and optimizing discrimination between the target nucleic acid and the variant thereof by manipulating design of the gRNA molecule, wherein said manipulation is done by: determining which sequence or secondary structure of the sgRNA yields optimum discrimination between the target nucleic acid and a variant thereof; and designing an sgRNA molecule accordingly.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A-1G show a quantitative massively parallel RNA profiling platform (FIG. 1A) RNA-CHAMP workflow. The sequenced mi-Seq chip with leftover fluorescent DNA strand is denatured and regenerated. RNA is generated by in vitro transcription. Fluorescent proteins are incubated in the chip and the fluorescent signal is then collected via TIRF microscopy. (FIG. 1B) A summary of a library used in this study. The top graph is a schematic for the library design. The left graph illustrates the type and the number of unique sequences. The right graph represents the number of clusters observed for unique sequences. (FIG. 1C) TIRF microscopy images after incubating with increasing concentrations of fluorescent Cas13d RNP. White circles indicate target clusters in the library. Red circles are T7 promoter scramble clusters that are not able to produce RNA. Orange circles are fiducial markers used for image alignment. (FIG. 1D) Quantification of fluorescent Intensities from TIRF microscopy images for indicated mismatch sequences. Intensities in all concentrations were fitted to the hyperbola equation. Error bars are the standard deviation of cluster intensities. (FIG. 1E) Correlation of two independent RNA-CHAMP experiments. The dashed lines are the detection limit of experiments. (FIG. 1F) Ranked order list of an RNA-CHAMP experiment. Dashed line represents the AABA of the perfect target. (FIG. 1G) Correlation of AABA measurements by RNA-CHAMP and biolayer interferometry. Error bars are the standard deviation of AABA.

FIGS. 2A-2F show that Cas13d lacks protospacer flanking sequence requirement (FIG. 2A) and (FIG. 2C) Heatmap of Normalized AABA in 5′ and 3′ PFS. Left, top, and right row are positions (FIG. 2A) 25, 24, and 23 (FIG. 2C) −1, −2, and −3 in the heatmap. Median normalized AABAs are shown in the graph. (FIG. 2B) and (FIG. 2D) MFE secondary structure of PFS sequences predicted by Vienna fold. Blue and black bases indicate the target region and constant region respectively. (FIG. 2E) BLI binding curves of highlighted PFS sequences in (FIG. 2A-2D). Grey lines are raw data curves and colored lines are fitted curves. (FIG. 2F) Normalized AABA of PFS sequences grouped by their base pairing count. The left and right graph are the sequence that have base pairs in positions 12-22 and 1-11 respectively. Error bars are the standard deviation of normalized AABA calculated by bootstrapping.

FIGS. 3A-3D show that RNA-CHAMP reveals sensitive binding region for mismatches sequences (FIG. 3A) Single mismatch analysis plot. The upper dash line is the matched target. Lower dashed is the detection limit of RNA-CHAMP. (FIG. 3B) Comparison of in vivo sensitivity region24 to RNA-CHAMP. (FIG. 3C) Double mismatches analysis. White regions indicate non-detectable sequences. (FIG. 3D) MFE secondary structure predicted by Vienna fold.

FIGS. 4A-4D show that proximal mismatches limit Cas13d cleavage activity (FIG. 4A). A schematic picture of the fluorescent cleavage assay (FIG. 4B) Fluorescent cleavage data of 10 mismatch sequences. (FIG. 4C) Initial slopes of the mismatch sequence. The slope is calculated from the 20 mins time point. Error bars are the standard deviation of two replicates. (FIG. 4D) Correlation of the cleavage slope and binding activity. Error bars of the slope are standard deviation calculated from two replicates and error bars of RNA-CHAMP are standard deviation calculated by bootstrapping.

FIGS. 5A-4E show modeling of Cas13d binding (FIG. 5A) Three components of modeling Cas13d binding. (FIG. 5B) Venn diagram of the Pearson r correlation from three main components. (FIG. 5C) Akaike information criterion of six models used in this study. (FIG. 5D) Hexagon plot correlates measured to predicted normalized AABAs from the model VI. (FIG. 5E) Weight penalty of base pairing and mismatches in the model VI.

FIG. 6A-B shows a model for Cas13d interaction (A) to various structured RNA (B).

FIG. 7A-D shows flag-tagged Tus was incubated in the chip and was labeled with anti-Flag-ATT0488 antibodies. About 90% of TerB-containing clusters were co-localized with Tus (FIG. 7A). To confirm the T7 RNAP-RNA complex is stably halted on the DNA, the ATTO647N-labeled DNA oligo was incubated complementary to the 5′ sequence of the target RNA. Results showed that almost ~90% of the RNAs were transcribed from promotor-containing clusters (FIG. 7B). Finally, purified SNAP-surface-488 labeled SNAP-dEsCas13d ribonucleoprotein (FIG. 7C) was incubated and imaged using a total internal reflection fluorescence (TIRF) microscopy. To prevent excessive crRNA hybridizing with the target RNA, the crRNA-Cas13d complex was purified by an additional size exclusion column and the RNP formation by native PAGE confirmed (FIG. 7D).

FIG. 8A-D shows that, similar to target A, a different target (target B) was also tested and little PFS preference was found except for a slight preference for non-G 3′PFS (FIG. 8A-B). The top 25% strong binding sequences were further combined in a logo plot and showed a very weak preference for non-G 3′PFS (FIG. 8C-8D).

FIG. 9A-C shows the single mismatch analysis was also perform for another target with complexed secondary structure (target C) and discovered a similar but slightly broader sensitivity region to mismatches at positions 9-20 (FIG. 9A). These results indicate that Cas13d binding is less tolerable to mismatches at the distal region of the target (FIG. 9B-C).

FIG. 10 shows mismatch versus deletion versus insertion for Target 1 and Target 3.

FIG. 11 shows point of care diagnostic with Cas13 lateral flow detection.

FIG. 12 shows SNP detection example: cancer allele detection. Foxp3 SNP (rs3761548) is involved in the gastric adenocarcinoma (GA) progression by influencing regulatory T cells function and cytokines. “A allele” demonstrated significantly greater risk of GA than “C allele”.

FIG. 13 shows SNP detection example: SARS-CoV2 variants detection.

FIG. 14 shows that Cas13d can tolerate mismatches at proximal positions. A high-throughput binding assay was performed. It was discovered that Cas13d tolerates mismatches at proximal positions (1-11). Negative AABA indicates the mismatch diminished protein binding affinity. Zero AABA indicates the mismatch doesn't affect protein binding.

FIG. 15 shows that cleavage activity of Cas13d was strongly diminished on proximal mismatches. The collateral cleavage activity was then tested on ten mismatches along the target sequence. Cas13d cleavage activity is strongly abolished when binding to proximal mismatch sequences (1-11).

### V. DETAILED DESCRIPTION

Before the present compounds, compositions, articles. devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

Unless defined otherwise, all technical and scientific terms used herein have the same meaning as commonly understood to one of ordinary skill in the art to which this disclosure belongs.

Ranges can be expressed herein as from “about” one particular value, and/or to “about” another particular value. By “about” is meant within 10% of the value, e.g., within 9, 8, 7, 6, 5, 4, 3, 2, or 1% of the value. When such a range is expressed, another aspect includes from the one particular value and/or to the other particular value. Similarly, when values are expressed as approximations. by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments and are also disclosed. Throughout the description and claims of this specification the word “comprise” and other forms of the word, such as “comprising” and “comprises,” means including but not limited to, and is not intended to exclude, for example, other additives, components, integers, or steps.

As used in the specification and claims, the singular form “a”, “an”, and “the” include plural references unless the context clearly dictates otherwise. For example, the term “an agent” includes a plurality of agents, including mixtures thereof.

As used herein, the terms “may,” “optionally.” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

As used herein, “nucleic acid” means a polynucleotide and includes a single or a double-stranded polymer of deoxyribonucleotide or ribonucleotide bases. Nucleic acids may also include fragments and modified nucleotides. Thus, the terms “polynucleotide”, “nucleic acid sequence” “nucleotide sequence” and “nucleic acid fragment” are used interchangeably to denote a polymer of RNA and/or DNA and/or RNA-DNA that is single- or double-stranded, optionally comprising synthetic, non-natural, or altered nucleotide bases. On occasion double-stranded DNA will be referred to “duplex DNA” or “dsDNA”. Nucleotides (usually found in their 5′-monophosphate form) are referred to by their single letter designation as follows: “A” for adenosine or deoxyadenosine (for RNA or DNA, respectively), “C” for cytosine or deoxycytosine, “G” for guanosine or deoxyguanosine, “U” for uridine, “T” for deoxythymidine, “R” for purines (A or G), “Y” for pyrimidines (C or T), “K” for G or T, “H” for A or C or T, “I” for inosine, and “N” for any nucleotide.

The term “genome” as it applies to a prokaryotic and eukaryotic cell or organism cells encompasses not only chromosomal DNA found within the nucleus, but organelle DNA found within subcellular components (e.g., mitochondria or plastid) of the cell.

“Open reading frame” is abbreviated ORF.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, or 90% sequence identity, up to and including 100% sequence identity (i.e., fully complementary) with each other.

The term “stringent conditions” or “stringent hybridization conditions” includes reference to conditions under which a probe will selectively hybridize to its target sequence in an in vitro hybridization assay. Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length. Typically, stringent conditions will be those in which the salt concentration is less than about 1.5 M Na ion, typically about 0.01 to 1.0 M Na ion concentration (or other salt(s)) at pH 7.0 to 8.3, and at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl. 1% SDS at 37° C., and a wash in 0.1×SSC at 60 to 65° C.

By “homology” is meant DNA sequences that are similar. For example, a “region of homology to a genomic region” that is found on the donor DNA is a region of DNA that has a similar sequence to a given “genomic region” in the cell or organism genome. A region of homology can be of any length that is sufficient to promote homologous recombination at the cleaved target site. For example, the region of homology can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases in length such that the region of homology has sufficient homology to undergo homologous recombination with the corresponding genomic region.

“Sufficient homology” indicates that two polynucleotide sequences have sufficient structural similarity to act as substrates for a homologous recombination reaction. The structural similarity includes overall length of each polynucleotide fragment, as well as the sequence similarity of the polynucleotides. Sequence similarity can be described by the percent sequence identity over the whole length of the sequences, and/or by conserved regions comprising localized similarities such as contiguous nucleotides having 100% sequence identity, and percent sequence identity over a portion of the length of the sequences.

As used herein, a “genomic region” is a segment of a chromosome in the genome of a cell that is present on either side of the target site or, alternatively, also comprises a portion of the target site. The genomic region can comprise at least 5-10, 5-15, 5-20, 5-25, 5-30, 5-35, 5-40, 5-45, 5-50, 5-55, 5-60, 5-65, 5-70, 5-75, 5-80, 5-85, 5-90, 5-95, 5-100, 5-200, 5-300, 5-400, 5-500, 5-600, 5-700, 5-800, 5-900, 5-1000, 5-1100, 5-1200, 5-1300, 5-1400, 5-1500, 5-1600, 5-1700, 5-1800, 5-1900, 5-2000, 5-2100, 5-2200, 5-2300, 5-2400, 5-2500, 5-2600, 5-2700, 5-2800, 5-2900, 5-3000, 5-3100 or more bases such that the genomic region has sufficient homology to undergo homologous recombination with the corresponding region of homology.

As used herein, “homologous recombination” (HR) includes the exchange of DNA fragments between two DNA molecules at the sites of homology. The frequency of homologous recombination is influenced by a number of factors. Different organisms vary with respect to the amount of homologous recombination and the relative proportion of homologous to non-homologous recombination. Generally, the length of the region of homology affects the frequency of homologous recombination events; the longer the region of homology, the greater the frequency. The length of the homology region needed to observe homologous recombination is also species-variable. In many cases, at least 5 kb of homology has been utilized, but homologous recombination has been observed with as little as 25-50 bp of homology. See, for example, Singer et al., (1982) Cell 31:25-33; Shen and Huang, (1986) Genetics 112:441-57; Watt et al., (1985) Proc. Natl. Acad. Sci. USA 82:4768-72, Sugawara and Haber, (1992) Mol Cell Biol 12:563-75, Rubnitz and Subramani, (1984) o/Cell Biol 4:2253-8; Ayares et al., (1986) Proc. Natl. Acad. Sci. USA 83:5199-203: Liskay et al., (1987) Genetics 115: 161-7.

“Sequence identity” or “identity” in the context of nucleic acid or polypeptide sequences refers to the nucleic acid bases or amino acid residues in two sequences that are the same when aligned for maximum correspondence over a specified comparison window.

The term “percentage of sequence identity” refers to the value determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. Useful examples of percent sequence identities include, but are not limited to, 50%, 55%, 60%, 65%, 70%, 75%, 80%, 85%, 90%, or 95%, or any percentage from 50% to 100%. These identities can be determined using any of the programs described herein.

Sequence alignments and percent identity or similarity calculations may be determined using a variety of comparison methods designed to detect homologous sequences including, but not limited to, the MegAlign™ program of the LASERGENE bioinformatics computing suite (DNASTAR Inc., Madison, WI). Within the context of this application it will be understood that where sequence analysis software is used for analysis, that the results of the analysis will be based on the “default values” of the program referenced, unless otherwise specified. As used herein “default values” will mean any set of values or parameters that originally load with the software when first initialized.

The “Clustal V method of alignment” corresponds to the alignment method labeled Clustal V (described by Higgins and Sharp, (1989) CABIOS 5:151-153; Higgins et al., (1992) Comput Appl Biosci 8: 189-191) and found in the MegAlign™ program of the

LASERGENE bioinformatics computing suite (DNASTAR Inc., Madison, WI). For multiple alignments, the default values correspond to GAP PENALTY=10 and GAP LENGTH PENALTY=10. Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences using the Clustal V program, it is possible to obtain a “percent identity” by viewing the “sequence distances” Table in the same program. The “Clustal W method of alignment” corresponds to the alignment method labeled Clustal W (described by Higgins and Sharp, (1989) CABIOS 5:151-153; Higgins et al., (1992) Comput Appl Biosci 8:189-191) and found in the MegAlign™ v6.1 program of the LASERGENE bioinformatics computing suite (DNASTAR Inc., Madison. WI). Default parameters for multiple alignment (GAP PENALTY=10. GAP LENGTH PENALTY=0.2, Delay Divergen Seqs (%)=30, DNA Transition Weight=0.5, Protein Weight Matrix=Gonnet Series, DNA Weight Matrix=IUB). After alignment of the sequences using the Clustal W program, it is possible to obtain a “percent identity” by viewing the “sequence distances” Table in the same program. Unless otherwise stated, sequence identity/similarity values provided herein refer to the value obtained using GAP Version 10 (GCG, Accelrys, San Diego, CA) using the following parameters:% identity and % similarity for a nucleotide sequence using a gap creation penalty weight of 50 and a gap length extension penalty weight of 3, and the nwsgapdna.cmp scoring matrix; % identity and % similarity for an amino acid sequence using a GAP creation penalty weight of 8 and a gap length extension penalty of 2, and the BLOSUM62 scoring matrix (Henikoff and Henikoff, (1989) Proc. Natl. Acad. Sci. USA 89: 10915). GAP uses the algorithm of Needleman and Wunsch, (1970) J Mol Biol 48:443-53, to find an alignment of two complete sequences that maximizes the number of matches and minimizes the number of gaps. GAP considers all possible alignments and gap positions and creates the alignment with the largest number of matched bases and the fewest gaps. using a gap creation penalty and a gap extension penalty in units of matched bases.

“BLAST” is a searching algorithm provided by the National Center for Biotechnology Information (NCBI) used to find regions of similarity between biological sequences. The program compares nucleotide or protein sequences to sequence databases and calculates the statistical significance of matches to identify sequences having sufficient similarity to a query sequence such that the similarity would not be predicted to have occurred randomly. BLAST reports the identified sequences and their local alignment to the query sequence. It is well understood by one skilled in the art that many levels of sequence identity are useful in identifying polypeptides from other species or modified naturally or synthetically wherein such polypeptides have the same or similar function or activity. Useful examples of percent identities include, but are not limited to, 50%, 55%, 60%, 65%, 70%, 75%, 80%, 85%, 90% or 95%, or any percentage from 50% to 100%. Indeed, any amino acid identity from 50% to 100% may be useful in describing the present disclosure, such as 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99%.

Polynucleotide and polypeptide sequences, variants thereof, and the structural relationships of these sequences can be described by the terms “homology”, “homologous”, “substantially identical”. “substantially similar” and“corresponding substantially” which are used interchangeably herein. These refer to polypeptide or nucleic acid sequences wherein changes in one or more amino acids or nucleotide bases do not affect the function of the molecule, such as the ability to mediate gene expression or to produce a certain phenotype. These terms also refer to modification(s) of nucleic acid sequences that do not substantially alter the functional properties of the resulting nucleic acid relative to the initial, unmodified nucleic acid. These modifications include deletion, substitution, and/or insertion of one or more nucleotides in the nucleic acid fragment. Substantially similar nucleic acid sequences encompassed may be defined by their ability to hybridize (under moderately stringent conditions, e.g., 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms to highly similar fragments, such as genes that duplicate functional enzymes from closely related organisms. Post-hybridization washes determine stringency conditions.

A “centimorgan” (cM) or “map unit” is the distance between two polynucleotide sequences, linked genes, markers, target sites, loci, or any pair thereof, wherein 1% of the products of meiosis are recombinant. Thus, a centimorgan is equivalent to a distance equal to a 1% average recombination frequency between the two linked genes, markers, target sites, loci, or any pair thereof.

An “isolated” or “purified” nucleic acid molecule, polynucleotide, polypeptide, or protein, or biologically active portion thereof, is substantially or essentially free from components that normally accompany or interact with the polynucleotide or protein as found in its naturally occurring environment. Thus, an isolated or purified polynucleotide or polypeptide or protein is substantially free of other cellular material, or culture medium when produced by recombinant techniques. or substantially free of chemical precursors or other chemicals when chemically synthesized. Optimally, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, in various embodiments, the isolated polynucleotide can contain less than about 5 kb. 4 kb. 3 kb. 2 kb. 1 kb. 0.5 kb. or 0.1 kb of nucleotide sequence that naturally flank the polynucleotide in genomic DNA of the cell from which the polynucleotide is derived. Isolated polynucleotides may be purified from a cell in which they naturally occur. Conventional nucleic acid purification methods known to skilled artisans may be used to obtain isolated polynucleotides. The term also embraces recombinant polynucleotides and chemically synthesized polynucleotides.

The term “fragment” refers to a contiguous set of nucleotides or amino acids. In one embodiment, a fragment is 2, 3, 4, 5, 6, 7 8, 9, 10, 11, 12, 13, 14, 15, 16, 17, 18, 19, 20, or greater than 20 contiguous nucleotides. In one embodiment, a fragment is 2, 3, 4, 5, 6, 7 8, 9, 10, 11, 12, 13, 14, 15, 16, 17, 18, 19, 20, or greater than 20 contiguous amino acids. A fragment may or may not exhibit the function of a sequence sharing some percent identity over the length of said fragment.

The terms “fragment that is functionally equivalent” and “functionally equivalent fragment” are used interchangeably herein. These terms refer to a portion or subsequence of an isolated nucleic acid fragment or polypeptide that displays the same activity or function as the longer sequence from which it derives. In one example, the fragment retains the ability to alter gene expression or produce a certain phenotype whether or not the fragment encodes an active protein. For example, the fragment can be used in the design of genes to produce the desired phenotype in a modified organism. Genes can be designed for use in suppression by linking a nucleic acid fragment, whether or not it encodes an active enzyme, in the sense or antisense orientation relative to a native promoter sequence.

“Gene” includes a nucleic acid fragment that expresses a functional molecule such as, but not limited to, a specific protein, including regulatory sequences preceding (5′ noncoding sequences) and following (3′ non-coding sequences) the coding sequence. “Native gene” refers to a gene as found in its natural endogenous location with its own regulatory sequences.

By the term “endogenous” it is meant a sequence or other molecule that naturally occurs in a cell or organism. In one aspect, an endogenous polynucleotide is normally found in the genome of a cell; that is, not heterologous.

An “allele” is one of several alternative forms of a gene occupying a given locus on a chromosome. When all the alleles present at a given locus on a chromosome are the same, that organism is homozygous at that locus. If the alleles present at a given locus on a chromosome differ, that organism is heterozygous at that locus.

“Coding sequence” refers to a polynucleotide sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include, but are not limited to, promoters, translation leader sequences, 5′ untranslated sequences, 3′ untranslated sequences, introns, polyadenylation target sequences, RNA processing sites, effector binding sites, and stem-loop structures.

A “mutated gene” is a gene that has been altered through human intervention.

Such a “mutated gene” has a sequence that differs from the sequence of the corresponding non-mutated gene by at least one nucleotide addition, deletion, or substitution. In certain embodiments of the disclosure, the mutated gene comprises an alteration that results from a guide polynucleotide/Cas endonuclease system as disclosed herein. A mutated organism is an organism comprising a mutated gene.

As used herein, a “targeted mutation” is a mutation in a gene (referred to as the target gene), including a native gene, that was made by altering a target sequence within the target gene using any method known to one skilled in the art, including a method involving a guided Cas endonuclease system as disclosed herein.

The terms “knock-out”, “gene knock-out” and “genetic knock-out” are used interchangeably herein. A knock-out represents a DNA sequence of a cell that has been rendered partially or completely inoperative by targeting with a Cas protein; for example, a DNA sequence prior to knock-out could have encoded an amino acid sequence, or could have had a regulatory function (e.g., promoter).

The terms “knock-in”, “gene knock-in, “gene insertion” and “genetic knock-in” are used interchangeably herein. A knock-in represents the replacement or insertion of a DNA sequence at a specific DNA sequence in cell by targeting with a Cas protein (for example by homologous recombination (HR). wherein a suitable donor DNA polynucleotide is also used) examples of knock-ins are a specific insertion of a heterologous amino acid coding sequence in a coding region of a gene, or a specific insertion of a transcriptional regulatory element in a genetic locus.

By “domain” it is meant a contiguous stretch of nucleotides (that can be RNA, DNA, and/or RNA-DNA-combination sequence) or amino acids.

“CRISPR” (Clustered Regularly Interspaced Short Palindromic Repeats) loci refers to certain genetic loci encoding components of DNA cleavage systems, for example, used by bacterial and archaeal cells to destroy foreign DNA (Horvath and Barrangou, 2010, Science 327: 167-170; WO2007025097, published 1 Mar. 2007). A CRISPR locus can consist of a CRISPR array. comprising short direct repeats (CRISPR repeats) separated by short variable DNA sequences (called spacers), which can be flanked by diverse Cas (CRISPR-associated) genes.

As used herein, an “effector” or “effector protein” is a protein that encompasses an activity including recognizing, binding to, and/or cleaving or nicking a polynucleotide target. An effector, or effector protein, may also be an endonuclease. The “effector complex” of a CRISPR system includes Cas proteins involved in crRNA and target recognition and binding. Some of the component Cas proteins may additionally comprise domains involved in target polynucleotide cleavage.

The term “Cas protein” refers to a polypeptide encoded by a Cas (CRISPR-associated) gene. A Cas protein includes proteins encoded by a gene in a cas locus and includes adaptation molecules as well as interference molecules. An interference molecule of a bacterial adaptive immunity complex includes endonucleases. A Cas endonuclease described herein comprises one or more nuclease domains. Contemplated herein are any Cas molecules that comprise a Rec3 clamp, as described below.

A Cas protein is further defined as a functional fragment or functional variant of a native Cas protein, or a protein that shares at least 30%, between 30% and 35%, at least 35%, between 35% and 40%, at least 40%, between 40% and 45%, at least 45%. between 45% and 50%, at least 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, at least 99%, between 99% and 100%, or 100% sequence identity with at least 50, between 50 and 100, at least 100, between 100 and 150, at least 150, between 150 and 200, at least 200, between 200 and 250, at least 250, between 250 and 300, at least 300, between 300 and 350, at least 350, between 350 and 400, at least 400, between 400 and 450. at least 500, or greater than 500 contiguous amino acids of a native Cas protein, and retains at least partial activity of the native sequence.

A “functional fragment”, “fragment that is functionally equivalent” and “functionally equivalent fragment” of a Cas endonuclease are used interchangeably herein. and refer to a portion or subsequence of the Cas endonuclease of the present disclosure in which the ability to recognize, bind to, and optionally unwind, nick or cleave (introduce a single or double strand break in) the target site is retained. The portion or subsequence of the Cas endonuclease can comprise a complete or partial (functional) peptide of any one of its domains.

The terms “functional variant”, “variant that is functionally equivalent” and “functionally equivalent variant” of a Cas endonuclease or Cas effector protein are used interchangeably herein, and refer to a variant of the Cas effector protein disclosed herein in which the ability to recognize, bind to, and optionally unwind, nick or cleave all or part of a target sequence is retained.

A Cas endonuclease may also include a multifunctional Cas endonuclease. The term “multifunctional Cas endonuclease” and “multifunctional Cas endonuclease polypeptide” are used interchangeably herein and includes reference to a single polypeptide that has Cas endonuclease functionality (comprising at least one protein domain that can act as a Cas endonuclease) and at least one other functionality, such as but not limited to, the functionality to form a complex (comprises at least a second protein domain that can form a complex with other proteins). In one aspect, the multifunctional Cas endonuclease comprises at least one additional protein domain relative (either internally, upstream (5′), downstream (3′), or both internally 5′ and 3′. or any combination thereof) to those domains typical of a Cas endonuclease.

As used herein, the term “guide polynucleotide”, relates to a polynucleotide sequence that can form a complex with a Cas endonuclease, including the Cas endonuclease described herein, and enables the Cas endonuclease to recognize, optionally bind to, and optionally cleave a DNA target site. The guide polynucleotide sequence can be an RNA sequence, a DNA sequence, or a combination thereof (a RNA-DNA combination sequence).

The terms “single guide RNA” and “sgRNA” are used interchangeably herein and relate to a synthetic fusion of two RNA molecules, a crRNA (CRISPR RNA) comprising a variable targeting domain (linked to a tracr mate sequence that hybridizes to a tracrRNA), fused to a tracrRNA (trans-activating CRISPR RNA).

The term “Cas endonuclease recognition domain” or “CER domain” (of a guide polynucleotide) is used interchangeably herein and includes a nucleotide sequence that interacts with a Cas endonuclease polypeptide. A CER domain comprises a (trans-acting) tracrNucleotide mate sequence followed by a tracrNucleotide sequence. The CER domain can be composed of a DNA sequence, a RNA sequence, a modified DNA sequence, a modified RNA sequence (see for example E1S20150059010A1. published 26 Feb. 2015), or any combination thereof.

As used herein, the terms “guide polynucleotide/Cas endonuclease complex”, “guide polynucleotide/Cas endonuclease system”,” guide polynucleotide/Cas complex”, “guide polynucleotide/Cas system” and “guided Cas system,” “Polynucleotide-guided endonuclease”, “PGEN” are used interchangeably herein and refer to at least one guide polynucleotide and at least one Cas endonuclease, that are capable of forming a complex, wherein said guide polynucleotide/Cas endonuclease complex can direct the Cas endonuclease to a DNA target site, enabling the Cas endonuclease to recognize, bind to, and optionally nick or cleave (introduce a single or double-strand break) the DNA target site. A guide polynucleotide/Cas endonuclease complex herein can comprise Cas protein(s) and suitable polynucleotide component(s) of any of the known CRISPR systems (Horvath and Barrangou, 2010, Science 327: 167-170; Makarova et al. 2015, Nature Reviews Microbiology Vol. 13: 1-15: Zetsche et al., 2015, Cell 163, 1-13; Shmakov et al., 2015, Molecular Cell 60, 1-13).

The terms “guide RNA/Cas endonuclease complex”, “guide RNA/Cas endonuclease system”, “guide RNA/Cas complex”, “guide RNA/Cas system”, “gRNA/Cas complex”, “gRNA/Cas system”. “RNA-guided endonuclease”, “RGEN” are used interchangeably herein and refer to at least one RNA component and at least one Cas endonuclease that are capable of forming a complex, wherein said guide RNA/Cas endonuclease complex can direct the Cas endonuclease to a DNA target site, enabling the Cas endonuclease to recognize, bind to, and optionally nick or cleave (introduce a single or double-strand break) the DNA target site.

The terms “target site”, “target sequence”, “target site sequence”, target DNA”, “target locus”, “genomic target site”. “genomic target sequence”, “genomic target locus” and “protospacer”, are used interchangeably herein and refer to a polynucleotide sequence such as, but not limited to, a nucleotide sequence on a chromosome, episome, a locus, or any other DNA molecule in the genome (including chromosomal, chloroplastic, mitochondrial DNA, plasmid DNA) of a cell, at which a guide polynucleotide/Cas endonuclease complex can recognize, bind to, and optionally nick or cleave. The target site can be an endogenous site in the genome of a cell, or alternatively, the target site can be heterologous to the cell and thereby not be naturally occurring in the genome of the cell, or the target site can be found in a heterologous genomic location compared to where it occurs in nature.

A “protospacer adjacent motif” (PAM) herein refers to a short nucleotide sequence adjacent to a target sequence (protospacer) that is recognized (targeted) by a guide polynucleotide/Cas endonuclease system described herein. The Cas endonuclease may not successfully recognize a target DNA sequence if the target DNA sequence is not followed by a PAM sequence. The sequence and length of a PAM herein can differ depending on the Cas protein or Cas protein complex used. The PAM sequence can be of any length but is typically 1, 2, 3, 4, 5, 6, 7, 8, 9, 10, 11, 12, 13, 14, 15, 16, 17, 18, 19, or 20 nucleotides long.

The term “variant” as used herein refers to a polypeptide or polynucleotide that differs from a reference polypeptide or polynucleotide, but retains essential properties. A typical variant of a polypeptide differs in amino acid sequence from another, reference, polypeptide. Generally, differences are limited so that the sequences of the reference polypeptide and the variant are closely similar overall (homologous) and, in many regions, identical. A variant and reference polypeptide may differ in amino acid sequence by one or more modifications (e.g., substitutions, additions, and/or deletions).

By “variant” or “fragment” is meant a functional fragment or functional variant of a native Cas protein, or a protein that shares at least 30%, between 30% and 35%, at least 35%, between 35% and 40%, at least 40%, between 40% and 45%, at least 45%, between 45% and 50%, at least 50%, 50%, between 50% and 55%, at least 55%, between 55% and 60%, at least 60%, between 60% and 65%, at least 65%, between 65% and 70%, at least 70%, between 70% and 75%, at least 75%, between 75% and 80%, at least 80%, between 80% and 85%, at least 85%, between 85% and 90%, at least 90%, between 90% and 95%, at least 95%, between 95% and 96%, at least 96%, between 96% and 97%, at least 97%, between 97% and 98%, at least 98%, between 98% and 99%, or at least 99% sequence identity to a parent Cas polypeptide. It is noted that “parent” and “native” are referred to alternatively herein, and have the same meaning, which is the naturally occurring Cas on which the variant or fragment thereof is based.

Throughout this application. various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. CRISPR/Cas Probe System

Disclosed herein is a method of detecting whether a sample comprises a target nucleic acid or a variant of the target nucleic acid, the method comprising: exposing the target nucleic acid to a CRISPR/Cas probe system; allowing the sgRNA to interact with the target nucleic acid or variant thereof in the presence of the Cas molecule and the nucleic acid probe. so that, upon hybridization of the crRNA with the target or variant thereof, the Cas molecule interacts with tracrRNA, and further wherein the Cas molecule cleaves the nucleic acid probe most efficiently when the crRNA is 100% complementary to the target nucleic acid; measuring signal from the nucleic acid probe. wherein one signal indicates that the target nucleic acid is 100% complementary to the crRNA, and wherein another signal indicates that a variant of the target nucleic acid is present. In some embodiments, the CRISPR/Cas probe system comprises: a single guide RNA (sgRNA) molecule, wherein said sgRNA comprises CRISPR RNA (crRNA) and tracrRNA, wherein said crRNA hybridizes with the target nucleic acid when the crRNA is 100% complementary to the target nucleic acid within a given region of the target nucleic acid, and wherein the crRNA also hybridizes with a variant of the target nucleic acid, wherein said variant comprises 1, 2, 3, or more mismatches within a given region of the target; a Cas molecule, wherein said Cas molecule cleaves nucleic acid when the crRNA is 100% complementary to the target nucleic acid within the given region. but does not cleave nucleic acid, or cleaves nucleic acid less efficiently, when the crRNA is hybridized with a variant which comprises one or two mismatches in the given region; and a nucleic acid probe, wherein said nucleic acid probe is cleavable by the Cas molecule, and further wherein the probe gives a different signal when cleaved compared to when it is not cleaved.

Also disclosed are platforms for detecting whether a sample comprises a target nucleic acid or a variant of the target nucleic acid, wherein the platform comprises: a CRISPR/Cas detection system and a detection means, whereby said detection means indicates whether a target nucleic acid or variant thereof is present. In some embodiments, said CRISPR/Cas detection system comprises: a single guide RNA (sgRNA) molecule, wherein said sgRNA comprises CRISPR RNA (crRNA) and tracrRNA, wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target: a Cas molecule, wherein said Cas molecule cleaves nucleic acid when the crRNA is 100% complementary to the target nucleic acid with the given region, but does not cleave nucleic acid, or cleaves nucleic acid less efficiently, when the crRNA is hybridized with a variant which comprises one or two mismatches in the given region; and a nucleic acid probe, wherein said nucleic acid probe is cleavable by the Cas molecule, and further wherein the probe gives a different signal when cleaved compared to when it is not cleaved.

Also disclosed is a method for optimizing discrimination between a target nucleic acid and at least one variant of the target nucleic acid during detection, the method comprising: determining a target nucleic acid and a variant of the target nucleic acid for detection, wherein discrimination between the target and the variant is desired; designing an sgRNA molecule, wherein said sgRNA comprises crRNA and tracrRNA, wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target; and optimizing discrimination between the target nucleic acid and the variant thereof by manipulating design of the gRNA molecule, wherein said manipulation is done by: determining which sequence or secondary structure of the sgRNA yields optimum discrimination between the target nucleic acid and a variant thereof; and designing an sgRNA molecule accordingly.

This invention allows the discrimination between target nucleic acids that are 100% complementary to the cRNA, meaning they contain no mismatches, and target nucleic acids which have 1, 2, 3, or more mismatches when hybridized with the crRNA. These mismatches can be at the proximal positions of the crRNA. By “proximal positions” is meant nucleic acid positions 1, 2, 3, 4, 5, 6, 7, 8, 9, 10, 11, 12, 13, 14, 15, 16, 17, 18, 19, or 20 from the proximal end of the crRNA. The “proximal end” is considered the 5′ end of the nucleic acid.

In some embodiments, the sample is from a subject. The sample may be a biological sample or an environmental sample. Biological samples may include, but are not necessarily limited to, blood, plasma, serum, urine, stool, sputum, mucous, lymph fluid, synovial fluid, bile, ascites, pleural effusion, seroma, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease, such as rheumatoid arthritis, osteoarthritis, gout or septic arthritis), or a swab of skin or mucosal membrane surface.

In certain embodiments, environmental samples may include. but are not necessarily limited to, samples obtained from a food sample. paper surface, a fabric, a metal surface, a wood surface, a plastic surface, a soil sample, a freshwater sample, a waste water sample, a saline water sample, or a combination thereof.

The sample can be used to determine whether, or what type, of infection or disease is present in an individual. This can be done by discriminating between, or detecting, single nucleotide polymorphisms (SNPs). In some embodiments, the disease state may be characterized by the presence or absence of an antibiotic or drug resistance or susceptibility gene or transcript or polypeptide. preferably in a pathogen or a cell.

In some embodiments, the infection may be caused by a virus, a bacterium, a fungus, a protozoan, or a parasite. In embodiments where the infection is viral, it may be caused by a DNA virus. In specific embodiments, the DNA virus may include, but is not necessarily limited to members of the Myoviridae. Podoviridae, Siphoviridae, Alloherpesviridae, Herpesviridae (including human herpes virus, and Varicella Zozter virus), Malocoherpesviridae, Lipothrixviridae, Rudiviridae, Adenoviridae, Ampullaviridae, Ascoviridae, Asfarviridae (including African swine fever virus), Baculoviridae, Cicaudaviridae, Clavaviridae, Corticoviridae, Fuselloviridae, Globuloviridae, Guttaviridae, Hytrosaviridae, Iridoviridae, Maseilleviridae, Mimiviridae, Nudiviridae, Nimaviridae, Pandoraviridae, Papillomaviridae, Phycodnaviridae, Plasmaviridae, Polydnaviruses, Polyomaviridae (including Simian virus 40, JC virus, BK virus), Poxviridae (including Cowpox and smallpox), Sphaerolipoviridae, Tectiviridae, Turriviridae, Dinodnavirus, Salterprovirus, or Rhizidovirus.

A viral infection may also be caused by a double-stranded RNA virus, a positive sense RNA virus, a negative sense RNA virus, a retrovirus, or a combination thereof. A viral infection may further be caused by a Coronaviridae virus, a Picornaviridae virus, a Caliciviridae virus, a Flaviviridae virus, a Togaviridae virus, a Bornaviridae, a Filoviridae, a Paramyxoviridae, a Pneumoviridae, a Rhabdoviridae, an Arenaviridae, a Bunyaviridae, an Orthomyxoviridae. or a Deltavirus. A viral infection may further be caused by Coronavirus, SARS, Poliovirus, Rhinovirus, Hepatitis A, Norwalk virus, Yellow fever virus, West Nile virus, Hepatitis C virus, Dengue fever virus, Zika virus, Rubella virus, Ross River virus, Sindbis virus, Chikungunya virus, Boma disease virus, Ebola virus, Marburg virus, Measles virus, Mumps virus, Nipah virus, Hendra virus, Newcastle disease virus, Human respiratory syncytial virus. Rabies virus, Lassa virus, Hantavirus, Crimean-Congo hemorrhagic fever virus, Influenza, or Hepatitis D virus.

In other embodiments, the infection may be bacterial in nature. The bacterium causing the bacterial infection may include, but is not necessarily limited to, an Acinetobacter species, an Actinobacillus species, an Actinomycetes species, an Actinomyces species, an Aerococcus species, an Aeromonas species, an Anaplasma species, an Alcaligenes species, a Bacillus species, a Bacteroides species, a Bartonella species, a Bifidobacterium species, a Bordetella species, a Borrelia species, a Brucella species, a Burkholderia species, a Campylobacter species, a Capnocytophaga species, a Chlamydia species, a Citrobacter species, a Coxiella species, a Corynbacterium species, a Clostridium species, an Eikenella species, an Enterobacter species, an Escherichia species, an Enterococcus species, an Ehlichia species, an Epidermophyton species, an Erysipelothrix species, an Eubacterium species, a Francisella species, a Fusobacterium species, a Gardnerella species, a Gemella species, a Hoemophilus species, a Helicobacter species, a Kingella species, a Klebsiella species, a Lactobacillus species, a Lactococcus species, a Listeria species, a Leptospira species, a Legionella species, a Leptospira species, a Leuconostoc species, a Mannheinia species, a Microsporum species, a Micrococcus species, a Moraxella species, a Morganell species, a Mobiluncus species, a Micrococcus species, Mvcobacterium species, a Mvcoplasm species, a Nocardia species, a Neisseria species, a Pasteurelaa species, a Pediococcus species, a Peptostreptococcus species, a Pityrosporum species, a Plesiononas species, a Prevotella species, a Porphyromonas species, a Proteus species, a Providencia species, a Pseudomonas species, a Propionibacteriums species, a Rhodococcus species, a Rickettsia species, a Rhodococcus species, a Serratia species, a Stenotrophomonas species, a Salmonella species, a Serratia species, a Shigella species, a Staphylococcus species, a Streptococcus species, a Spirillum species, a Streptobacillus species, a Treponema species, a Tropheryma species, a Trichophyton species, an Ureaplasma species, a Veillonella species, a Vibrio species, a Yersinia species, a Xanthomonas species, or combination thereof.

In other embodiments, the infection may be fungal, and may be caused by fungi such as, but not necessarily limited to, Aspergillus, Blastonyces, Candidiasis, Coccidiodomycosis, Cryptococcus neoformans, Cryptococcus gatti, sp. Histoplasma sp. (such as Histoplasma capsulatum), Pneumocystis sp. (such as Pneumocystis jirovecii), Stachybotrys (such as Stachybotrys chartarum), Mucroymcosis, Sporothrix, fungal eye infections ringworm, Exserohilum, Cladosporium, Geotrichum, Saccharomyces, a Hansenula species, a Candida species, a Kluyveromyces species, a Debaryomyces species, a Pichia species, a Penicillium species, a Cladosporiun species, a Byssochlamys species or a combination thereof.

In other embodiments, the infection may be caused by a protozoan, such as Euglenozoa, a Heterolobosea, a Diplomonadida, an Amoehozoa, a Blastocystic, an Apicomplexa, or combination thereof.

In other embodiments, the infection may be caused by a parasite. such as, but not necessarily limited to, Trypanosoma cruzi (Chagas disease). T. brucei gambiense, T. brucei rhodesiense, Leishmania braziliensis, L. infantum, L. mexicana, L. major, L. tropica, L. donovani, Naegleria fowleri, Giardia intestinalis (G. lamblia, G. duodenails), canthamoeba caste llanii, Balamuthia madrillaris, Entamoeba histolytica, Blastocystic hominis, Babesia microti, Cryptosporidium parvum, Cyclospora cavetanensis, Plasmodium falciparum, P. vivax, P. ovale, P. malariae, and Toxoplasma gondii, or a combination thereof.

In some embodiments, the Cas molecule has non-specific cleavage activity. By “non-specific cleavage activity” is meant that the Cas molecule does not require a specific sequence in order cleave the nucleic acid. In some embodiments, the Cas molecule lacks protospacer flanking sequence (PFS) recognition, which is a trait of some Cas systems, such as Cas13d

In some embodiments, the Cas molecule is Cas13. Cas13 includes four subtypes, Cas13a-d (types VI A-D). Cas13a, Cas13b, and Cas13d have a “bilobed” architecture comprising recognition (REC) and nuclease (NUC) lobes. In some embodiments, the Cas molecule is Cas13d.

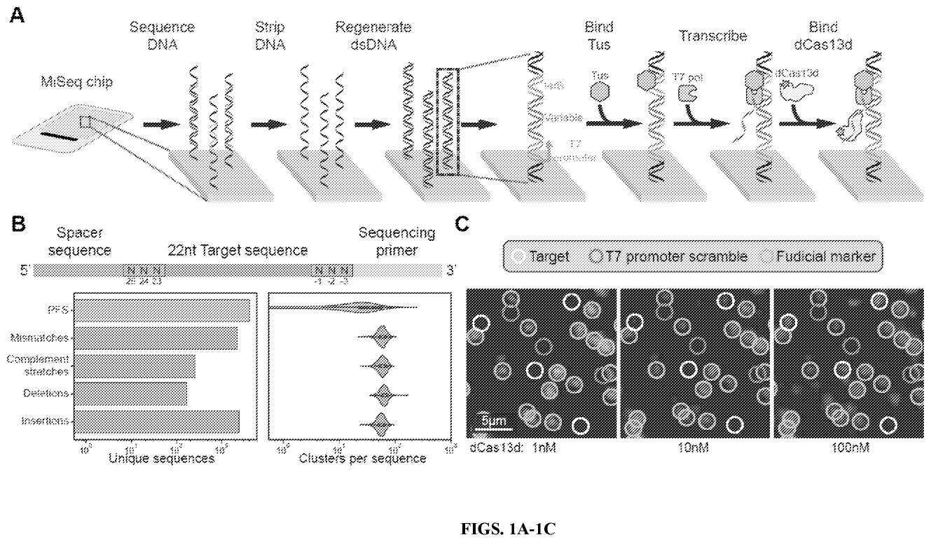

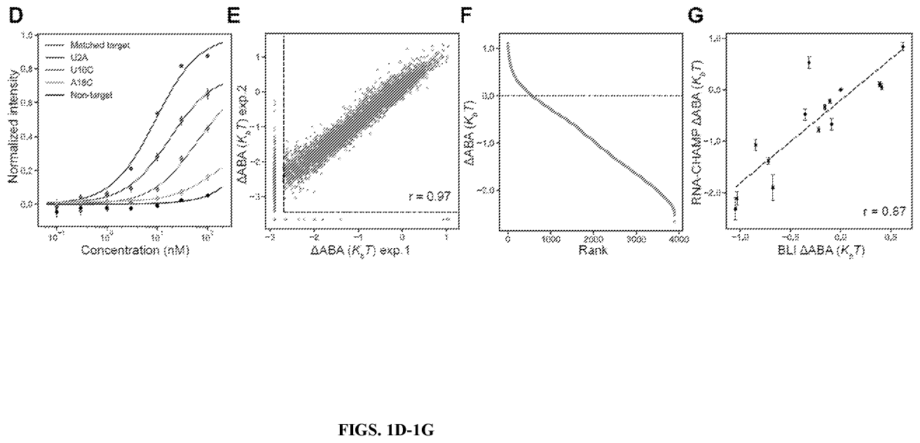

By “given region” in the claims is meant a specific component of the target nucleic acid, which is less than the entire target nucleic acid. For example, the given region can be at a proximal end of the target nucleic acid in relation to the tracrRNA. This can be seen, for example, in FIG. 1A. This region can be 1, 2, 3, 4, 5, 6, 7, 8, 9, 10, 11, 12, 13, 14, or 15 nucleotides long or longer. In one specific embodiment, this regions is 1-11 nucleotides long. In some embodiments, the given region is a distal spacer region of the target nucleic acid when it is bound by the Cas complex. This can be due to the fact that RNA secondary structures can affect crRNA hybridization kinetics. For example, the RNA secondary structures can occlude the distal region (for example, positions 11-22 when Cas13d is used) of the target sequence, which can strongly diminish the binding affinity of the Cas complex. Mismatches in this same region (positions 11-22) of the target nucleic acid can largely reduce the binding affinity (Example 1).

Methods of detection of nucleic acid hybridization using a probe are known to those of skill in the art. It is contemplated that the target nucleic acid could be captured and sequenced, or merely the detection of the nucleic acid could be accomplished. In some embodiments, the probe can comprise a fluorophore and quencher molecule. In some embodiments, the probe comprises a metal nanoparticle.

In certain embodiments, a device can be employed which allows for the rapid detection of a target nucleic acid or variant thereof. This device, for example, can comprise a flow strip. For instance, a lateral flow strip allows for RNAse detection by color. The RNA reporter is modified to have a first molecule (such as for instance FITC) attached to the 5′ end and a second molecule (such as for instance biotin) attached to the 3′ end (or vice versa). The lateral flow strip is designed to have two capture lines with anti-first molecule (e.g., anti-FITC) antibodies hybridized at the first line and anti-second molecule (e.g., anti-biotin) antibodies at the second downstream line. As the reaction flows down the strip, uncleaved reporter binds to anti-first molecule antibodies at the first capture line, while cleaved reporters liberate the second molecule and allow second molecule binding at the second capture line. Second molecule sandwich antibodies, for instance conjugated to nanoparticles, such as gold nanoparticles, binds any second molecule at the first or second line and result in a strong readout/signal (e.g. color). As more reporter is cleaved, more signal can accumulate at the second capture line and less signal appears at the first line.

Lateral support substrates may be located within a housing (see for example, “Rapid Lateral Flow Test Strips” Merck Millipore 2013). The housing may comprise at least one opening for loading samples and a second single opening or separate openings that allow for reading of detectable signal generated at the first and second capture regions.

Specific binding-integrating molecules can be used with this system, and can comprise binding pairs, for example. Such binding pairs are known to those skilled in the art and include, but are not limited to, antibody-antigen pairs, enzyme-substrate pairs. receptor-ligand pairs, and streptavidin-biotin. In addition to such known binding pairs, novel binding pairs may be specifically designed. A characteristic of binding pairs is the binding between the two members of the binding pair.

The presence of target nucleic acid can cause a stronger cleavage reaction by Cas, and therefore a more intense signal, compared to a variant of the target nucleic acid. By “stronger cleavage reaction” is meant 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or 2, 3, 4, 5, 6, 7, 8, 9, or 10 times, or more, stronger cleavage reaction of the target nucleic acid than the variant (wherein the variant comprises 1, 2, 3, or more mismatches). In some cases, the more mismatches, the weaker the cleavage reaction, so that one mismatch has a stronger cleavage reaction than 2 mismatches, and 2 mismatches have a stronger cleavage reaction than 3, etc. By “more intense signal” is meant 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, or 2, 3, 4, 5, 6, 7, 8, 9, or 10 times, or more, of an intense signal when the target nucleic acid is detected than the variant nucleic acid (wherein the variant comprises 1, 2, 3, or more mismatches).

In some embodiments, the method of any preceding aspect is high throughput. In some embodiments, the method of any preceding aspect is quantitative. In some embodiments, RNA chip-hybridized affinity-mapping platform (RNA-CHAMP) is used to carry out the method. In some embodiments, the method is done in multiplex so that more than one variant of a single target nucleic acid can be detected simultaneously, or nearly simultaneously (within 1, 2, 3, 4, 5, 6, 7, 8, 9, or 10 minutes apart). Multiplex analysis of samples enables large-scale detection of samples, reducing the time and cost of analyses. In some embodiments, said means of measuring further comprises a readout of a signal from the probe. In some embodiments, said readout of signal from the probe comprises ELISA. In some embodiments, said readout is a point of care diagnostic. In some embodiments, said point of care diagnostic is a handheld test. In some embodiments, said means of measuring comprises a computer system. wherein said computer system interprets the signal. In some embodiments, said computer system displays results.

When the method of optimizing discrimination is used, the sequence of the crRNA can be designed to maximize discrimination between a target nucleic acid and a variant thereof, wherein the variant comprises 1, 2, 3, or more mismatches. In some embodiments, the secondary structure of the gRNA is designed to maximize discrimination. For example, the sequences or secondary structure can be maximized with regard to the distal spacer region of the target nucleic acid or variant thereof. This is described above.

### VI. EXAMPLES

### Example 1. Introduction

Cluster regularly interspaced palindromic repeats (CRISPR) and CRISPR associated (Cas) proteins provide adaptive immune systems against foreign genetic elements in prokaryotes. Class II CRISPR-Cas systems currently include Type II, Type V, and recently discovered Type VI. Class II systems rely on a single effector for nucleic acid interference and are widely adopted as various genetic engineering tools due to their simplicity. Among all Class II systems, Type VI CRISPR-Cas13 is the only one that exclusively targets RNA. The CRISPR-Cas13 system is currently classified into Cas13a, Cas13b, Cas13c, Cas13d, Cas13λ and Cas13Y. All Cas13 subtypes have been shown to possess crRNA-dependent target RNA (cis-cleavage) and a non-specific RNA cleavage activity (trans-cleavage). Both RNA cleavage activities are mediated by two higher eukaryotes and prokaryotes nucleotide-binding domains (HEPN). Due to its distinct RNA cleavage capabilities, Cas13s are harnessed for a wide variety of RNA engineering tasks.

Type VI-D effectors (Cas13d) are smaller than other Cas13s and lack protospacer flanking sequences (PFS) requirement. Cas13d is shown to have better RNA knockdown efficiency in mammalian cell lines and is also effective in various animal models. Recently, Cas13d is also used for nucleic acid detection and shown to have comparable activity to Cas13a detection tools. Catalytic inactive Cas13d is also a superb tool in targeting specific RNA by fusing with fluorescent protein or functional domains for RNA tracking, editing, modification, and splicing. However, Cas13d binding and cleavage specificity was not yet fully characterized.

This study describes an RNA chip-hybridized affinity-mapping platform (RNA-CHAMP) for massively parallel profiling of RNA-protein interactions. RNA-CHAMP is based on a previously described chip-hybridized affinity-mapping platform (CHAMP) leverages the sequenced Illumina chips for DNA-protein profiling. With fluorescently imaging and a dedicated computational imaging analysis pipeline. CHAMP is an ideal platform for massively parallel DNA-protein interaction profiling. To profile RNA-protein interaction, this study adopted the high-throughput sequencing-RNA affinity profiling (HiTS-RAP) to transcribe and tether the RNA on its cognate DNA by using a roadblock to stall T7 polymerase. HiTS-RAP uses a discontinued Illumina sequencer and required additional modification on the machine. RNA-CHAMP is based on an Illumina miSeq machine which is widely available and does not require additional modification.

RNA-CHAMP and other biochemical methods were used to profile the interaction between the Eubacterium siraeum Cas13d-crRNA complex and the target RNA. RNA-CHAMP revealed that Cas13d binding affinity was minimally affected by PFS. However, RNA secondary structures that occlude the distal region (positions 11-22) of the target sequence strongly diminish the binding affinity of Cas13d. This study also found that mismatches in the similar “seed” region (positions 11-22) of the target largely reduce the binding affinity. These results show that Cas13d can search and recognize the target RNA by solvent-exposed distal spacer region. Surprisingly, targets with mismatches in positions 1-11 were not affected in binding affinity but were failed to activate Cas13d nuclease activity. These results show that a combined mechanism by preventing both binding and cleavage to achieve high specificity when encountering mismatches in different positions. Altogether, this study shows that Cas13d binding and cleavage were affected by various ways which have strong implications in developing accurate RNA engineering tools.

### Example 2. Results

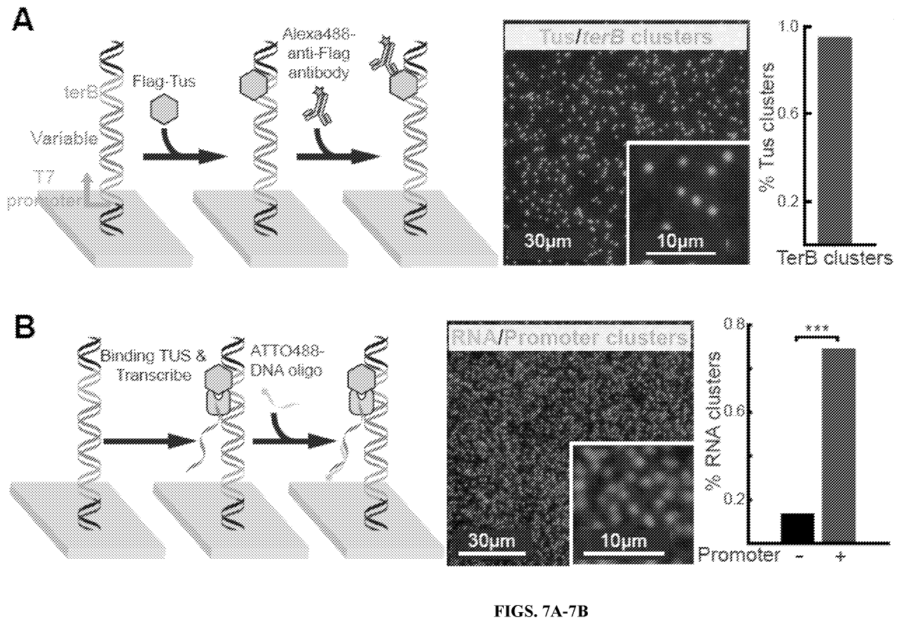

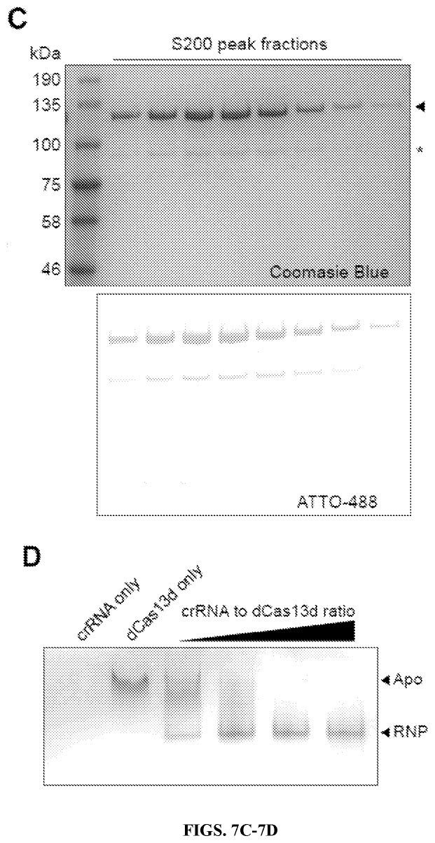

Overview of RNA-CHAMP workflow. RNA-CHAMP is a quantitative high-throughput protein-RNA profiling platform that utilized a sequenced Illumina MiSeq chip. Illumina platform allows massively parallel sequencing of millions of clusters and each cluster is spatially separated and registered. The physical coordinates of each sequenced DNA cluster are reported during the next-generation sequencing (NGS). After sequencing, the chip with leftover fluorescent nucleotides is stripped and regenerated into dsDNA (FIG. 1A). To convert the dsDNA into tethered RNAs, this study adopted the sequence design of high-throughput sequencing—RNA affinity profiling (HiTS-RAP), which operates on discontinued Illumina Genome Analyzer IIx. However, RNA-CHAMP is built on a widely available MiSeq platform and microscopy, without further modification on MiSeq machine. The library was comprised of a transcription template flanked by T7 RNA polymerase (RNAP) promoter and Tus binding site (TerB) (FIG. 1A). Tus, an E. coli transcription terminator protein, was added to bind to TerB site as a roadblock for halting T7 RNAP (FIG. 1A). To first confirm Tus binding on DNA, Flag-tagged Tus was incubated in the chip and was labeled with anti-Flag-ATT0488 antibodies. About 90% of TerB-containing clusters were co-localized with Tus (FIG. 7A). However, there were few Tus clusters not co-localized with TerB-containing clusters could be due to the unidentified sequence from the sequencer. T7 RNAP was incubated for on-chip transcription which was halted by Tus with transcribed RNA on its cognate DNA (FIG. 1A). To confirm the T7 RNAP-RNA complex is stably halted on the DNA, the ATTO647N-labeled DNA oligo was incubated complementary to the 5′ sequence of the target RNA. Results showed that almost ~90% of the RNAs were transcribed from promotor-containing clusters (FIG. 7B). Finally, purified SNAP-surface-488 labeled SNAP-dEsCas13d ribonucleoprotein (FIG. 7C) was incubated and imaged using a total internal reflection fluorescence (TIRF) microscopy (FIG. 1A). Fluorescent images were processed by the previously described CHAMP software pipeline to map the fluorescent clusters to the Illumina sequencing data. The target library comprises three randomized bases on both 5′ and 3′ protospacer flanking sequencing, and up to two bases of mismatches, insertions, and deletions (FIG. 1B).

The binding intensity of dEsCas13d (hereafter referred to as “Cas13d”) was measured across ~1000-fold concentration gradient in twofold or threefold increments from 0.125 to 128 nM or 0.1 to 100 nM respectively (FIGS. 1C, 1D). To prevent excessive crRNA hybridizing with the target RNA, the crRNA-Cas13d complex was purified by an additional size exclusion column and the RNP formation by native PAGE confirmed (FIG. 7D). At each concentration, Cas13d was incubated in the chip, washed out, and imaged. Scramble T7 promoter and non-complementary RNA clusters were included as background and negative control (FIG. 1C). Fluorescent intensities of clusters across all concentrations were subtracted with background intensity and fitted with hyperbola equation to determine the apparent K_{d} values (FIG. 1D). Duplicate experiments were performed for all libraries and resulted in a range of correlation coefficient of 0.78-0.97 (FIG. 1E), indicating high reproducibility of this method. This study set the detection limit from apparent K_{d} of 1 nM to 128 nM which is the highest concentration used in RNA-CHAMP. Apparent K_{d} values within the detection limit in both replicates were used in the following analyses. Δ Apparent binding affinity (AABAs) was calculated from the log transformation of the apparent K_{d} of perfect target divided by the alerted sequences. Depending on the target sequences, up to 4000 sequences in an experiment were able to be captured within detection limits (FIG. 1F). To further validate RNA-CHAMP measurements, bio-laver interferometry was used to measure the kinetics of 15 sequences from the library. Apparent binding affinity (ABA) normalized from a mismatched target to the matched target for both measurements have a correlation coefficient of 0.88 (FIG. 1G). Overall, a rapid and quantitative platform were established for protein-RNA interaction profiling.

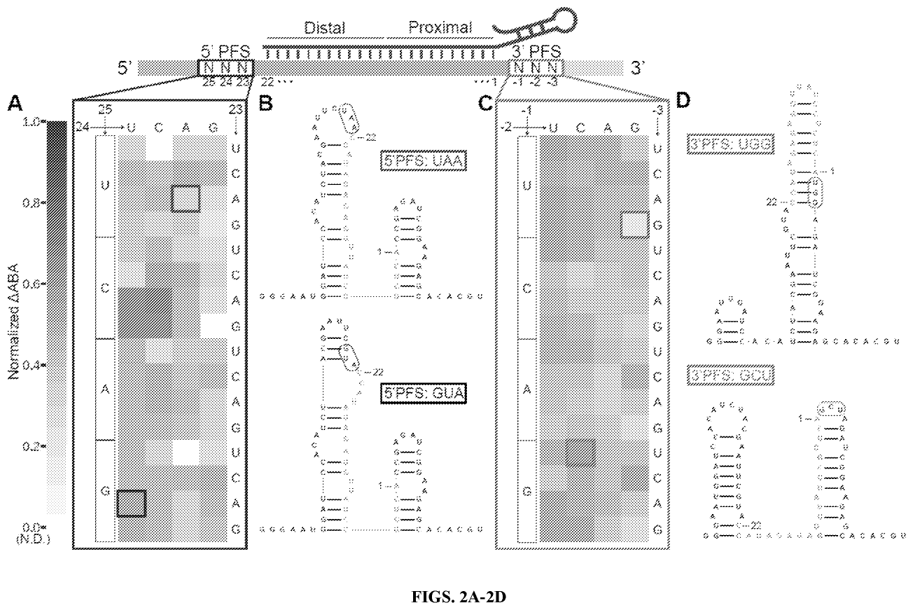

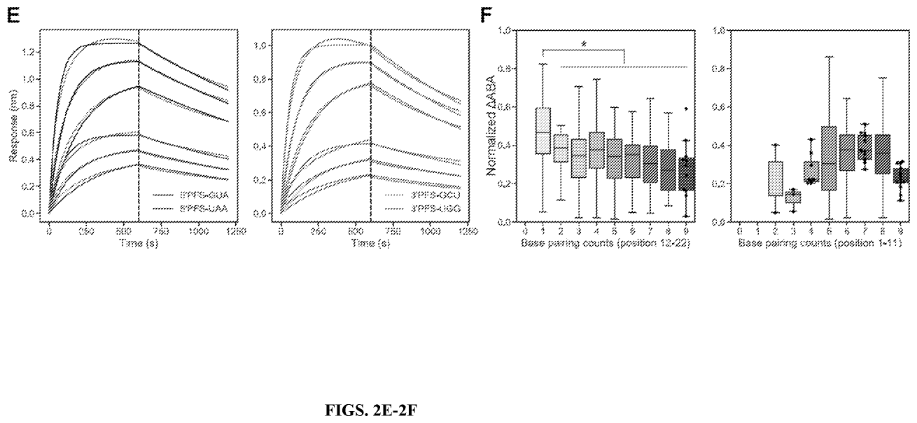

Cas13d lack of obvious PFS requirement. CRISPR-Cas13 systems require varying PFS for RNA targeting. LshCas13a prefers a non-G 3′-PFS, whereas BzCas13b favors non-G 5′-PFS and 3′PFS of NNA or NAN. However, commonly used CRISPR-Cas13 systems such as LshCas13a, PspCas13b, RfxCas13d (CasRx) do not have PFS requirements. Previous studies used high-throughput in vivo assays and biochemical assays for PFS analysis mainly on Cas13d cleavage. Here, the PFS requirement for Cas13d binding was further investigated via RNA-CHAMP. PFS library consists of three random nucleotides on the 5′ and 3′ end of the 22 nt target sequence. 4096 unique sequences were profiled, resulting in ~1400 sequences with detectable ABAs in target A. These results showed that there was no single nucleotide at specific positions that were strongly favored or depleted, suggesting that RNA binding of Cas13d does not require specific PFS (FIGS. 2A and 2C).

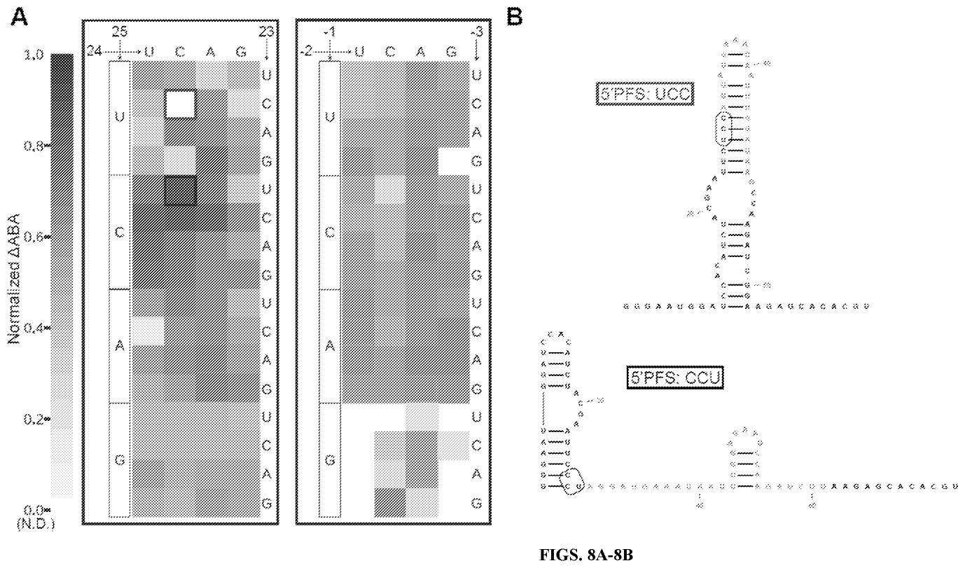

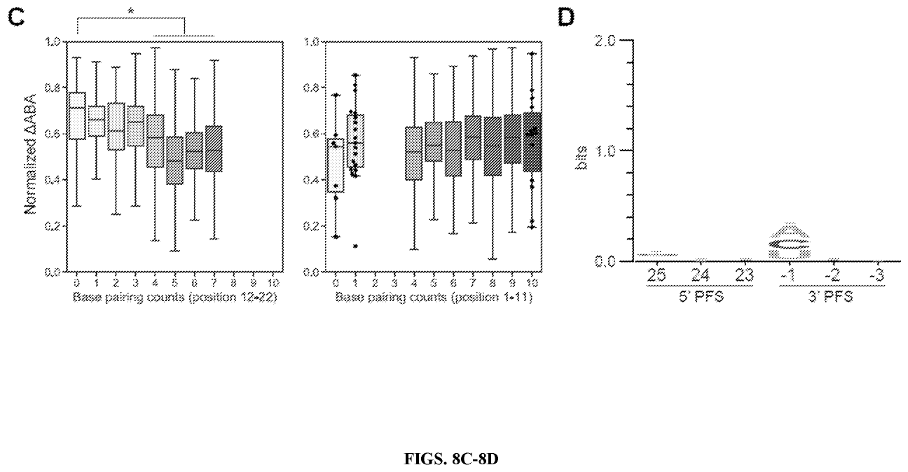

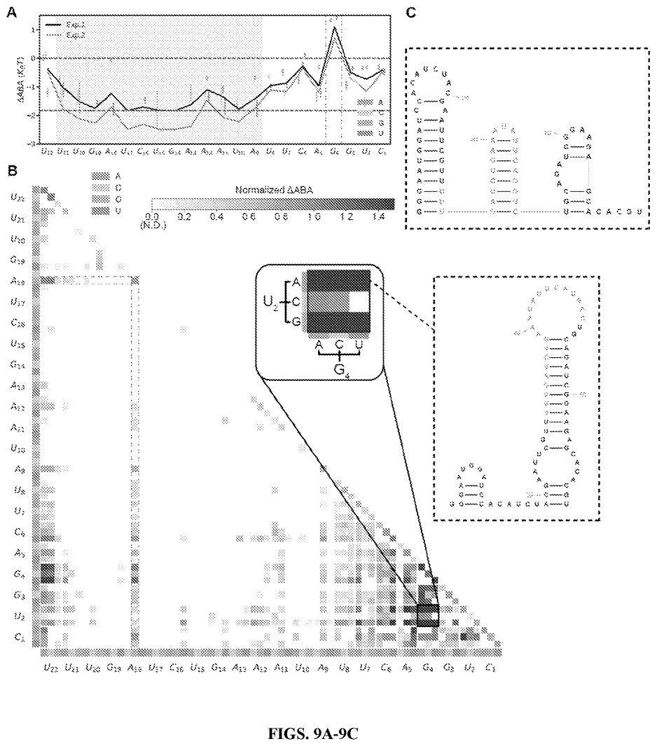

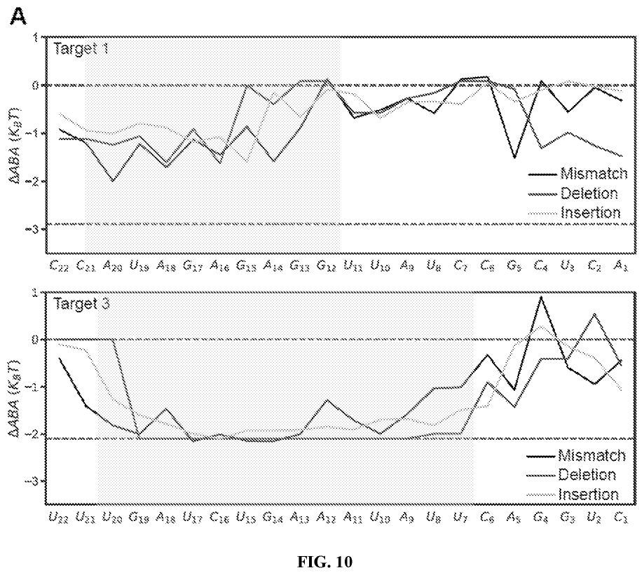

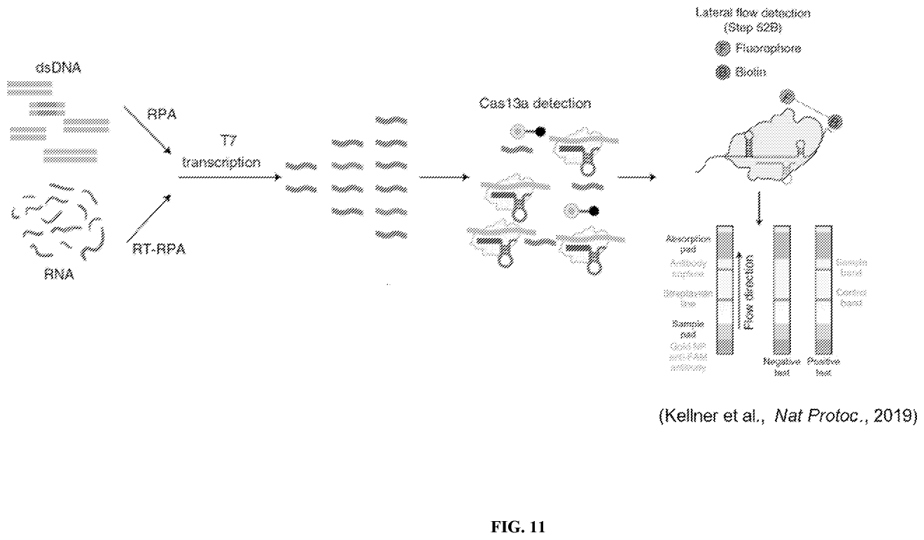

Similar to target A, a different target (target B) was also tested and found little PFS preference except for a slight preference for non-G 3′PFS (FIG. 8A). The top 25% strong binding sequences were further combined in a logo plot and showed a very weak preference for non-G 3′PFS (FIG. 8D).

Interestingly, the binding affinity of strong and weak binding sequences in these library members could differ up to ~3-fold. This is because that the RNA secondary structure is one of the determining factors for binding of Cas13d. Minimum free energies (MFE) predicted by Vienna fold showed a slight correlation between measured binding affinity. The secondary structures of these sequences were also looked at, a stretch of open RNA was observed at the distal region (position 11-22) is usually found in strong binding sequences (FIGS. 2A-2D). 5′PFS-GUA forms a stem with the 5′ constant region and exposed position 19-22 which leads to ~2-fold stronger binding than 5′PFS-UAA, which those positions were formed within the stem.

Similarly, 3′PFS-GCU forms a stem with the 5′ constant region and exposed position 14-20. Again, exposed distal nucleotides lead to stronger binding affinity than 3′PFS-UGG. To further confirm these observations, bio-layer interferometry was used to measure the binding kinetics of these sequences. It was found that weak binding sequences have similar off-rate but slower on-rate than strong binding sequences (FIG. 2E). Also, the BLT results from those weak binding sequences were reaching their maximum responses around 2-fold less than their strong binding sequences. This indicated that RNAs were less accessible for Cas13d, indicating that RNA structure is precluding Cas13d binding. To get a broader view of how local accessibility affects Cas13d binding, the sequences were grouped by base pairing counts in the proximal and distal region. A clear trend of decreasing binding affinity with increasing base pairing counts was found in the distal region of the RNA, whereas no statically significant trend was identified in the proximal region of the RNA (FIG. 2F). Taken together, this study found that Cas13d does not have significant PFS requirements which are similar to previous studies. Additionally, this study also showed that Cas13d tends to bind to distally accessible RNA

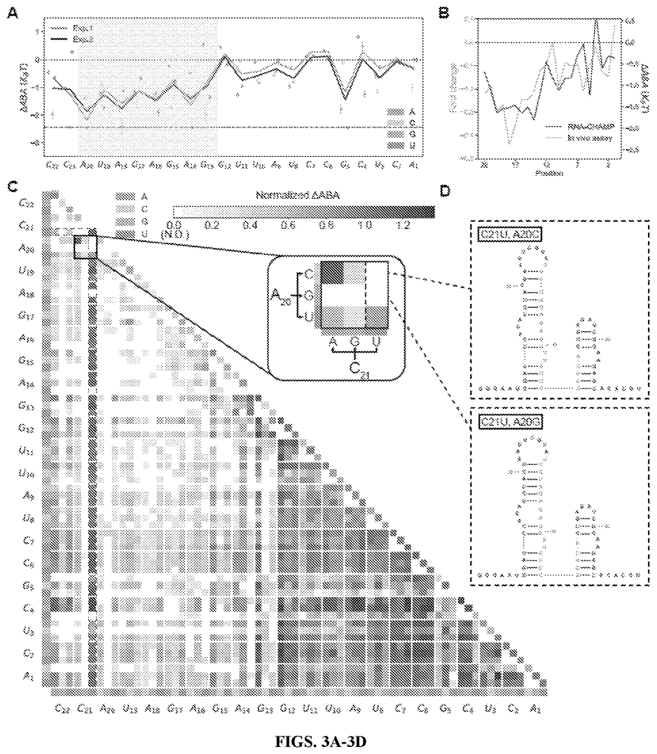

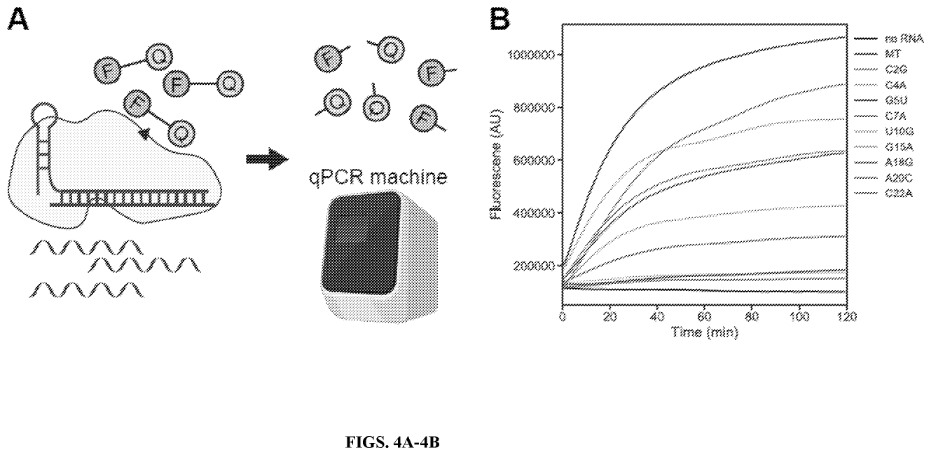

Binding specificity of Cas13d. To determine the possible “seed” region and binding specificity of Cas13d, a library comprised of single and double mismatches along the 22-nt target sequence was constructed. Single mismatch analysis showed a weaker binding affinity at position 13-20. In contrast, sequences with single mismatches at positions 1-12 have little to no effect on the binding affinity compared to the matched target. To exclude sequence-specific biases, the single mismatch analysis was also perform for another target with complexed secondary structure (target C) and discovered a similar but slightly broader sensitivity region to mismatches at positions 9-20 (FIG. 9A). These results indicate that Cas13d binding is less tolerable to mismatches at the distal region of the target. The existing Cas13d cleavage dataset also showed that mismatches in position 15-21 strongly diminished CasRx knockdown activity in human cells (FIG. 3B).

A distal sensitivity region was observed in double mismatch analysis. The binding affinity of Cas13d is strongly diminished when both mismatches were located in positions 13-20. Conversely, the binding affinity is almost unaffected if both mismatches occurred in positions 1-12. Together, Cas13d has low binding tolerance to distal mismatch sequences. However, Cas13d is highly tolerable to proximal mismatch sequences. This should be carefully considered to prevent unwanted off-target effect when using Cas13d as a therapeutic and diagnostic tool. Although, it's difficult to deconvolute the effect of the mismatch and structure accessibility in this particular analysis. This study discovered a few “stripes” in the double mismatches analysis that were strongly affected by RNA structures. Mismatch C21G has a distal inaccessible structure that was bound weakly by Cas13d and out of the detection limit. However, the structure was not changed in C21U and the mismatch also forms G-U wobble base pair with the crRNA, resulting in a similar binding affinity to the matched target. A second mismatch (A20C or A20G) addition to C21U results in an undetectable signal of Cas13d binding is possibly due to inaccessible distal RNA regions (FIG. 3D). Additional mismatch A20U to C21U retains the same structure as the matched target and the binding affinity was only slightly affected. Overall, Cas13d binding is sensitive to distal mismatches as well as the accessibility of the local distal RNA region.

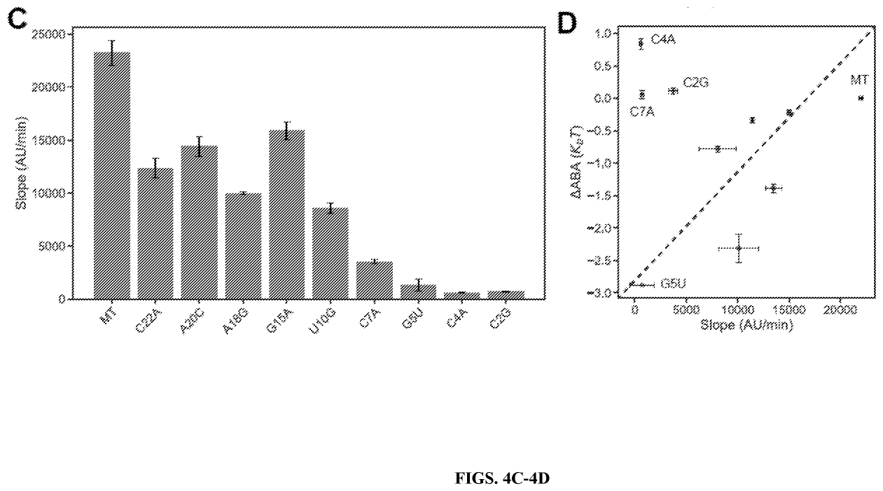

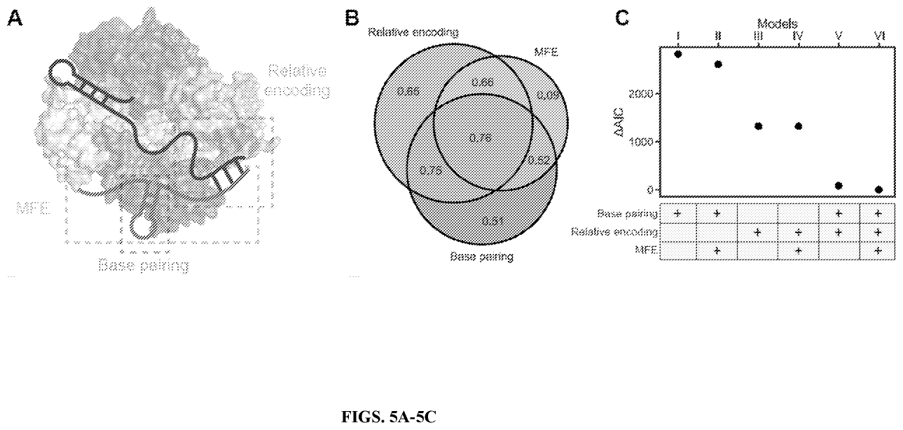

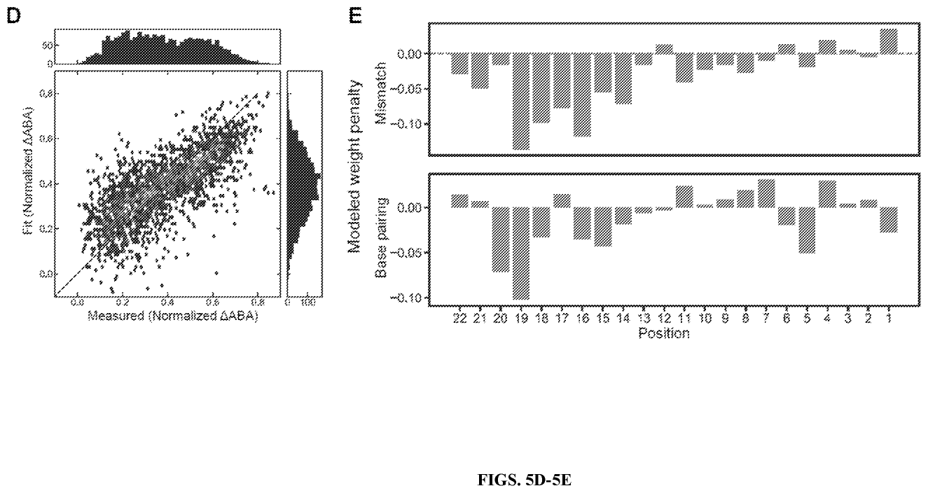

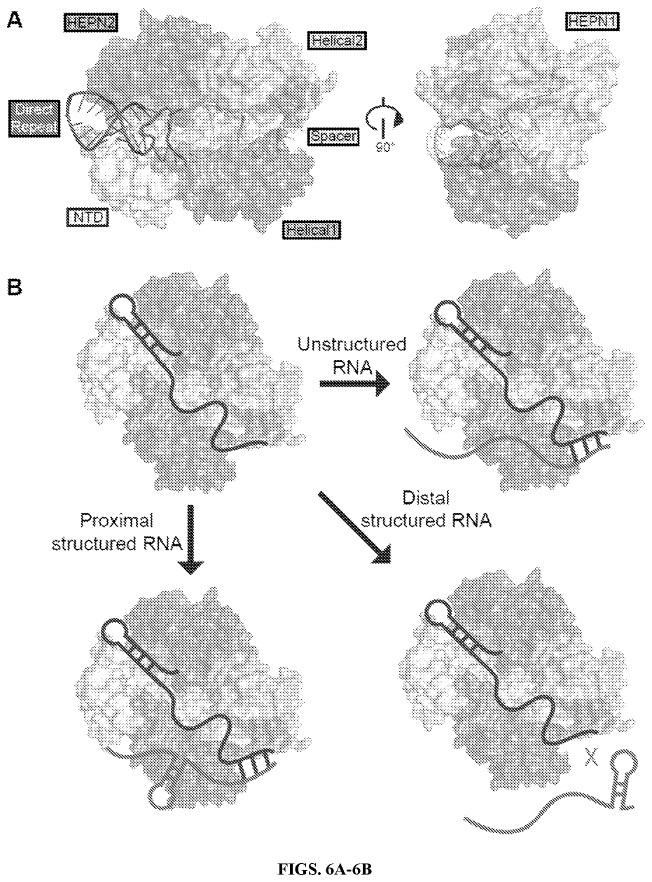

Proximal mismatches diminish the cleavage activity of Cas13d. Since Cas13d is widely used in CRISPR diagnostics, how mismatch sequences affect the collateral cleavage activity of Cas13d was tested. To measure the collateral cleavage activity, active Cas13d-crRNA RNP was incubated with various target RNAs and fluorescent RNA reporters (FIG. 4A). Once Cas13d RNP binds to the target RNA, activated cleavage activity can cleavage the RNA reporter and release the fluorescent dye from the quencher. The fluorescent signal was then collected by a qPCR machine.

The data revealed that the fluorescent signal was strongly diminished in mismatches C2G, C4A, G5U, and C7A (FIGS. 4B and 4C). The binding affinity of mismatch sequences (C2G. C4A, and C7A) were not affected in RNA-CHAMP and BLI assays. When the binding affinity was correlated to the initial slope of the cleavage curve, most of the sequences correlates well except C2G, C4A, and C7A (FIG. 4D). This indicate that Cas13d binding can tolerate mismatches in those position but were not able to activate the HEPN-nuclease activity. Previous structural study Cas13d showed that there were several amino acids interacting with the crRNA-target duplexes, including positions 3-6. C2G, C4A, and C7A disrupts the amino acid-RNA duplex interface that block the conformational changes required for active HEPN-nuclease activity. Overall, it was found that Cas13d binding is not sensitive to mismatch sequences in positions 1-11, whereas Cas13d cleavage is dramatically weakened. In contrast, Cas13d still retains cleavage activity, albeit slightly reduced, to mismatches sequences in positions 12-22.

Previous reports have shown that the binding and cleavage activities of Cas13a were decoupled. Cas13a has a position 9-14 “seed” region for binding and a position 5-8 region critical for cleavage activity. Here, this study revealed a similar mechanism in Cas13d but slightly shifted binding and cleavage sensitivity region. These discoveries indicating that the binding and cleavage activity is a universal characteristic in other Cas13 systems.

A biophysical model for Cas13d binding specificity. Next, a simple biophysical model was built to better quantify how sequence alterations, accessibility, and overall secondary structure would affect Cas13d binding. There were three major components in this model (FIG. 5A). First, Minimum free energy (MFE) of the full length 73-nt RNA MFE structure was predicted by Vienna fold and described the overall complexity of the RNA structure. Second, base pairing counts along the 22-nt target RNA indicate the accessibility of the target RNA. This component has a total of 22 parameters for each position to reflect whether if the RNA is base paired with other nucleotides in the predicted MFE structure. Third, the type of alteration and position along the 22-nt target RNA describes the mispaired bases along the RNA duplex. There is a total of 9 types of alteration in every position, including four nucleotide mismatches, four nucleotide insertions, and one deletion.

Based on these biochemically related parameters, the model's performance was evaluated by information loss via Akaike information criterion (AIC) and Pearson correlation. Surprisingly, Model I and II which is purely based on structural parameters gives a Pearson correlation of r~0.5 was close to the more comprehensive Model III (FIG. 5B, 5C) indicating that structural features play a vital role in determining Cas13d binding affinity. However, an extra MFE only slightly improves the model performance further suggesting that local structure with the target RNA is more important than the full-length RNA structure (FIG. 5B, 5C). Finally, when combining structural features with mispairing features, Model VI improves the correlation to r>0.75 and highlights the structural and position features in binding specificity (FIG. 5B-5E) The model again shows the binding of Cas13d is strongly penalized to altered distal sequences.

Similarly, the model also highlights that Cas13d binding is penalized when base pairing occurred in a similar region, especially positions 19 and 20.

### Example 3. Methods

126. Protein Expression and Purification. Catalytic inactive EsCas13d (dEsCas13d, R295A/H300A/R849A/H854A) was cloned into pET-based plasmid with 6×His/Twin-Strep/SUMO/SNAP N-terminal fusion. The plasmid was transformed into BL21 star (DE3) cells (Thermo Fisher). Cells were inoculated in LB containing carbenicillin to OD600 0.7 and induced with 200 mM IPTG at 18° C. for 18 hours. Cells were then pelleted, resuspended in lysis buffer (50 mM HEPES pH 7.4, 500 mM NaCl, 1 mM EDTA, 5% glycerol, 0.1% Tween® 20, 1 mM DTT, cOmplete™-EDTA-free protease inhibitor cocktail (Sigma Aldrich), 1 mg/ml lysozyme, 2.5 U/ml DNaseI, 2.5 U/ml salt active nuclease), and lysed completely by sonication. Clarified lysate was applied to a Strep-Tactin® Superflow® gravity column (IBA Life Sciences). The Strep-Tactin® resin was washed with 20 column volumes (CVs) of wash buffer (50 mM HEPES pH 7.4, 500 mM NaCl, 5% glycerol, 1 mM DTT), eluted with 5 CVs of elution buffer (50 mM HEPES pH 7.4, 500 mM NaCl, 10% glycerol, 5 mM D-desthiobiotin, 1 mM DTT), and then concentrated by a spin concentrator (Amicon® 30 kDa cutoff Ultra 15, Millipore). Concentrated samples were incubated with SUMO protease for tag cleavage and SNAP-Surface® 488 dye (NEB) if used for RNA-CHAMP experiments at 4° C. for 20 hours. Samples were then further purified by a size-exclusion column (Superdex 200™ Increase 10/300 GL, GE Life Sciences) using SEC buffer (50 mM Tris-HCL pH 7.5, 500 mM NaCl. 10% glycerol, 2 mM DTT). For RNP purification, purified dCas13d was incubated with 6-fold excess of crRNA (IDT) at 37° C. for 1 hour in RNP buffer (50 mM Tris-HCL pH 7.5, 100 mM NaCl, 6 mM MgCl₂, 1 mM DTT), and again subjected to size-exclusion column (Superdex 200™ Increase 10/300 GL, GE Life Sciences) to further separate the RNP from the crRNA by using RNP SEC buffer (50 mM Tris-HCL pH 7.5, 150 mM NaCl, 1 mM MgCl₂, 10% Glycerol. 1 mM DTT). RNP fractions were pooled, spin concentrated by a spin concentrator (Amicon® 30 kDa cutoff Ultra 15, Millipore), and flash frozen in liquid nitrogen and store at −80° C.

Library design and preparation. PFS DNA libraries were PCR amplified (Q5® High-Fidelity 2×Master Mix) from oligonucleotides with three randomized bases on either ends of the target ordered from IDT. PCR reaction also extends the amplicons with Illumina adapters and barcodes for later MiSeq sequencing. Similar to PFS libraries, target libraries contain various alterations were PCR amplified from pooled custom oligonucleotides from Synthego. Amplified DNA library were sequenced by MiSeq (Illumina) using MiSeq reagent kit v3 (150-cycle).

RNA-CHAMP. RNA-CHAMP instrument set up is identical to previous published CHAMP. Briefly, MiSeq chip were placed on the microscope for image collection through a custom-built microscope adapter with integrated microfluidics inlet and outlet. Flow rate was controlled via an automated syringe pump (KD scientific) and is kept at 100 μl/min for all procedures. Details for all components are available in Github.

After sequencing, MiSeq chip with leftover fluorescent nucleotides were denatured with 500 μl 0.1N NaOH and washed with 500 μl TE buffer (10 mM Tris-HCL, 500 mM EDTA). The chip is then incubated with 500 nM regeneration primers in hybridization buffer (5×SSC, 0.1% Tween® 20) for 5 mins at 85° C., cooled to 65° C. over 10 mins. cooled to 40° C. over 30 mins, and hold at 40° C. for 10 mins. During the last 10 mins at 40° C., the chip was wash with 1 mL wash buffer to remove unannealed primers.

Biolayer Interferometry. Binding kinetics were assessed by using biolayer interferometry on an Octet RED96e (FortéBio). Recombinant ACE2 and spike variants were diluted in binding buffer. Biotinylated RNA was immobilized on strepavidin biosensors (FortéBio). The biosensors were then immersed in the indicated concentrations of Cas13d RNP complex for 600 s as the association step and transferred into binding buffer for 600 s to measure Cas13d RNP dissociation. The signal was acquired from a reference sensor without any spike protein. This trace was treated as a baseline and subtracted from all other association and dissociation curves. The kₒₙ, k_{off}, and Kd values were calculated from global fitting to all the binding curves by using Octet data analysis software vi 1.1.

### Collateral Cleavage Fluorescent Assay

Computational Modeling. The Apparent Binding Affinity (ABA) rates for mutated sequences kt off the target base strand k! were modeled using a linear combination of relevant features (Model 1) shown below.

$$\text{?} = \text{?} * \text{?} ( i , v ) + \text{?} * {f}_{D} ( i , 0 ) + \text{?} {c}_{i , v} * {f}_{R} ( i , v ) + d * g ( k ) + C$$

$$\text{?} \text{indicates text missing or illegible when filed}$$

The value of N is equal to the length of the target RNA strand, and S represents the set of possible target mutation bases {A, C, G, U}. The generalized function ƒ5 is an indicator function which evaluates as:

$${f}_{x} ( i , v ) = \{ \begin{matrix}1 , & if oper x used to transfrom {k}_{t} to k \\ 0 , & Otherwise\end{matrix}$$

For a simplified example take the base strand k! to be the sequence AUUG and let the modified strand k be UUC. Then the set of operations used to transform AUUG to UUC is

$$\{ ( Del , 1 ) , ( Rep , 4 , C ) \}$$

Which means that the first base was deleted, and the third base was replaced with C. Thus ƒ1(0, 0) and ƒ4(4, C) would evaluate to 1 and all other inputs for ƒ5 would evaluate to 0. In testing ƒ5 is assumed to be a k-sparse function with k≤4.

The function g outputs the minimal free energy in kcal/mol of sequence k using ViennaRNA libraries. The choice to use the ViennaRNA free energy predictor as an extra variable in the model was because the ViennaRNA had a non-negligible correlation with ABA rates (FIG. 1) and is relatively orthogonal to the coordinate-wise operations gathered from ƒ. The weights of the terms a_{i,v}, bᵢ, and c_{i,v} fit using the data and represent the penalties of each operational transformation on kt.

ABA rates that are longer than the furthest time point of measuring cannot be evaluated effectively. Thus, after experimentation inputs with measured ABA larger than the highest detectible ABA rates were filtered out before training the model. The outputs of the model were also constrained using a “bandpass filter” function:

$$B ( x ) = \{ \begin{matrix}x , & x \leq m \\ m , & x > m\end{matrix}$$

Where m is the constant representing the furthest reasonable ABA rate which for the models was determined to be 6.

Ridge regression was used to determine the weights of the parameters to fit to training set ABA measurements. Ridge regression is a variant of linear regression which attempts to minimize the training loss value of the expression:

$$\text{?} {( M ( k ) - {k}_{ABA} )}^{2} + λ \text{?} {β}^{2}$$

$$\text{?} \text{indicates text missing or illegible when filed}$$

M is defined to be the model for predicting ABA with all the weights β being values in set X={aᵢ, bᵢ, cᵢ, d}. The predicted values from Model M are compared to the measured ABA values k_{ABA}: the smaller the absolute difference between the two the greater model's accuracy. Ridge regression helps maintain robustness of linear models and prevents overfitting by penalizing arbitrarily large weights. The parameter λ at which the weight values in the model appear to stabilize is around 1 which was used throughout all models. Extending the Feature Space: Encoding

A helpful tool to create more expressive and accurate models from gathered features is a function to encode the parameter space. Here an encoding is defined as a function E such that:

$$E ( k ) = ( {x}_{1} , {x}_{2} , {x}_{3} … {x}_{n} , )$$

Where k is the given input string of RNA bases, and the x$∈ [symbol] is the set of feature values. A natural encoding of the string k is provided by the binary function ƒ defined above. A simple algorithm can be used to match an RNA sequence k to an encoding E(k), mainly E(k)ᵢ=1 if

$$( T ( i \% 9 ) , ⌊ \frac{i}{9} ⌋ ∈ ops ( k )$$

otherwise 0. The value of

$$⌊ \frac{i}{9} ⌋$$

determines the position of the operation; ops(k) is the set of operations to transform kᵣ to k; and T(k) is defined as the operation table:

| Value | Operation |
|---|---|
| 0 | Delete |
| 1 | Insert A |
| 2 | Insert C |
| 3 | Insert G |
| 4 | Insert U |
| 5 | Replace A |
| 6 | Replace C |
| 7 | Replace G |
| 8 | Replace U |

The above encoding is defined as a relative encoding the value of the encoded string is relative to base strand. This contrasts with an absolute encoding which is defined as

$${E ( k )}_{i} = 1 if ( {T}_{abs} ( i \% 4 ) , ⌊ \frac{i}{4} ⌋ ) otherwise 0$$

T_{abc} is defined as the simplified lookup table:

| Value | Base |
|---|---|
| 0 | A |
| 1 | C |
| 2 | G |
| 3 | U |

The advantages of relative encoding over absolute encoding are interpretability—the sparsity of the relative encoding leads to clear interpretations for how a mutation will effect ABA rates; insertion and deletion accuracy one deletion at the beginning of the sequence effects only one parameter in the relative encoding but could have arbitrarily large effect on an absolute sequence; and applicability-most problem statements in this space attempt to create small permutations in a protein or RNA sequence to maintain some property but which to augment stability, dispersion, etc. One disadvantage of such an encoding is that it requires ~2× more variables and thus has higher potential to overfit on small datasets.

### Example 4. CRISPR-Based Single Nucleotide Polymorphism Detection Via Programmed Mismatch

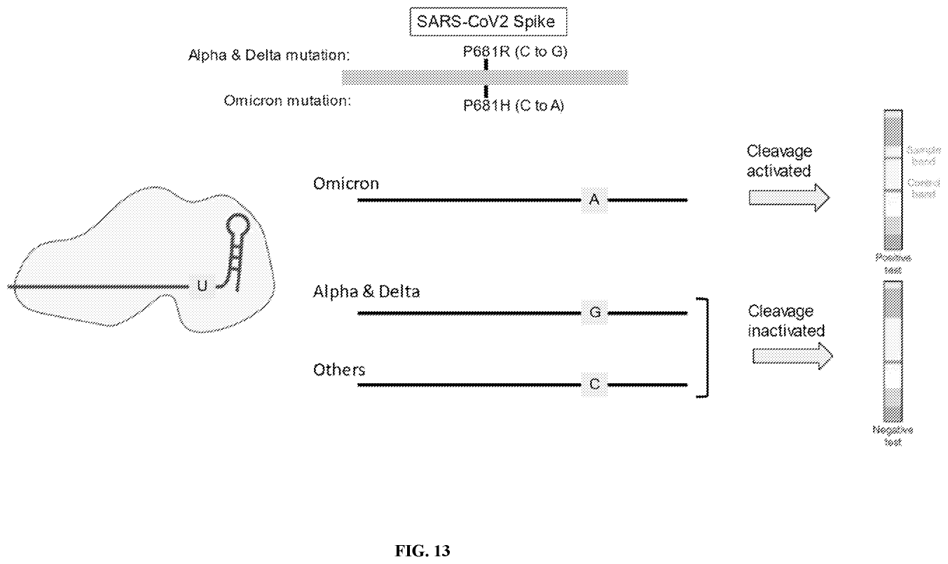

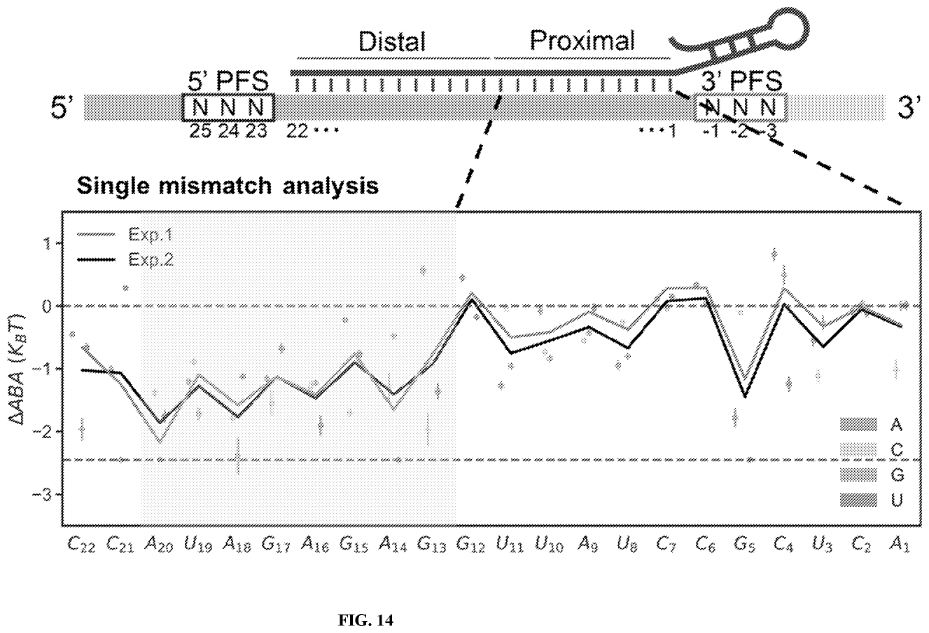

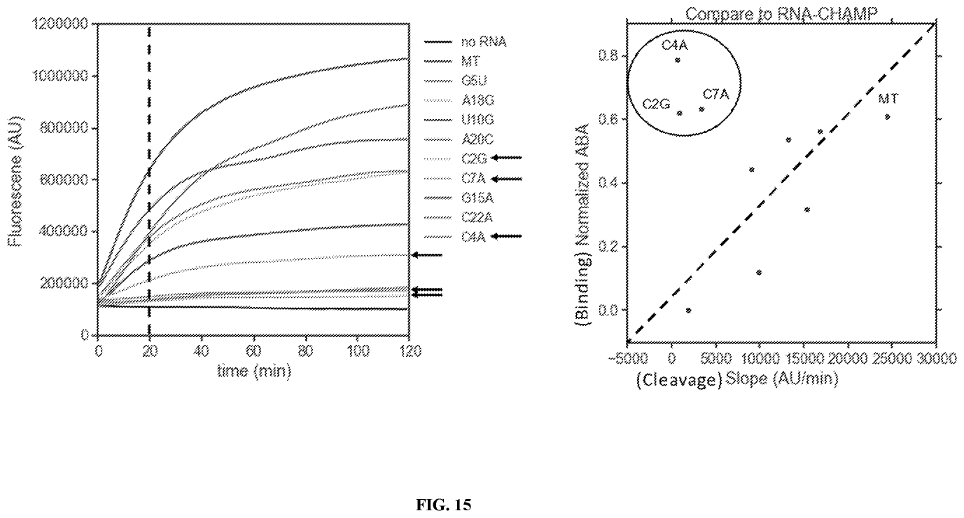

Current CRISPR-based diagnostics are focusing on detecting if a specific DNA/RNA is presented in the sample. The approach shown herein can detect specific DNA/RNA variants in single nucleotide resolution. Examples can be found in FIGS. 14 and 15. CRISPR-based diagnostics are the only point-of-care method for quick and accurate SNP detection. See FIG. 13. The global CRISPR gene detection and diagnostic market is expected to account for USD 4,708,89 million by 2028.

Advantages of Cas13d: Cas13d enzymes are approximately 20% smaller than Cas13a, b effectors. Cas13d does not require a Protospacer Flanking Sequence (PFS). These characteristics present an advantage for protein production and flexible targeting.

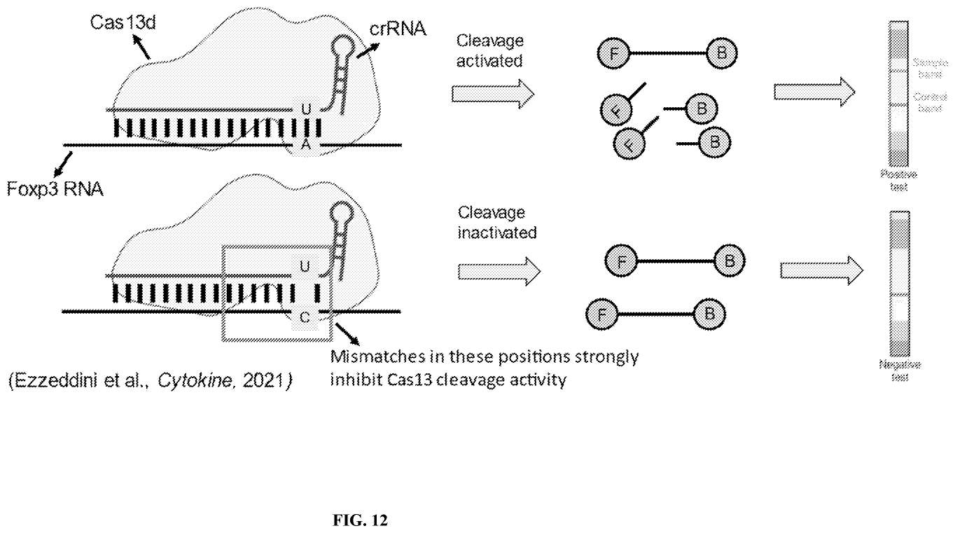

SNP is a single position in DNA that has various nucleotides among individuals. SNPs were shown to involve many diseases including heart diseases, diabetes, cancer, and neurodegenerative diseases. Common methods for SNP detection usually require lab equipment and/or time consuming. SNP detection example can be found in FIG. 12, which shows cancer allele detection using Cas13.

1. A method of detecting whether a sample comprises a target nucleic acid or a variant of the target nucleic acid, the method comprising:
a. exposing the target nucleic acid to a CRISPR/Cas probe system, wherein said system comprises:
i. a single guide RNA (sgRNA) molecule, wherein said sgRNA comprises CRISPR RNA (crRNA) and tracrRNA, wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target;
ii. a Cas molecule, wherein said Cas molecule cleaves nucleic acid when the crRNA is 100% complementary to the target nucleic acid with the given region, but does not cleave nucleic acid, or cleaves nucleic acid less efficiently, when the crRNA is hybridized with a variant which comprises one or two mismatches in the given region; and
iii. a nucleic acid probe, wherein said nucleic acid probe is cleavable by the Cas molecule, and further wherein the probe gives a different signal when cleaved compared to when it is not cleaved;
b. allowing the sgRNA to interact with the target nucleic acid or variant thereof in the presence of the Cas molecule and the nucleic acid probe, so that, upon hybridization of the crRNA with the target or variant thereof, the Cas molecule interacts with tracrRNA, and further wherein the Cas molecule cleaves the nucleic acid probe most efficiently when the crRNA is 100% complementary to the target nucleic acid;
c. measuring signal from the nucleic acid probe, wherein one signal indicates that the target nucleic acid is 100% complementary to the crRNA, and wherein another signal indicates that a variant of the target nucleic acid is present.

2. (canceled)

3. The method of claim 1, wherein the variant of the target nucleic acid is a single nucleotide polymorphism (SNP).

4. The method of claim 1, wherein the Cas molecule has non-specific cleavage activity

5. The method of claim 1, wherein the Cas molecule lacks protospacer flanking sequence recognition.

6. The method of claim 1, wherein the Cas molecule is Cas13.

7. The method of claim 6, wherein the Cas molecule is Cas13d.

8. The method of claim 1, wherein the given region is at a proximal end in relation to the tracrRNA

9. (canceled)

10. The method of claim 8, wherein the given region is 1-11 nucleotides long.

11. The method of claim 1, wherein the given region is a distal spacer region.

12. The method of claim 1, wherein the target and/or variant thereof comprises secondary structure, wherein said secondary structure affects crRNA hybridization kinetics.

13. The method of claim 1, wherein the probe comprises a fluorophore and quencher molecule or a metal nanoparticle.

14. (canceled)

15. The method of claim 1, wherein, in step (c), the presence of target nucleic acid causes a stronger cleavage reaction by Cas, and therefore a more intense signal, compared to a variant of the target nucleic acid.

16. (canceled)

17. The method of claim 1, wherein said method is quantitative.

18. The method of claim 1, wherein RNA chip-hybridized affinity-mapping platform (RNA-CHAMP) is used to carry out the method.

19. (canceled)

20. The method of claim 1, wherein more than one target nucleic acid can be detected simultaneously.

21. The method of claim 20, wherein multiple variants of multiple target nucleic acids can be detected simultaneously.

22.-27. (canceled)

28. A platform for detecting whether a sample comprises a target nucleic acid or a variant of the target nucleic acid, wherein the platform comprises:
a. a CRISPR/Cas detection system, wherein said CRISPR/Cas detection system comprises:
i. a single guide RNA (sgRNA) molecule, wherein said sgRNA comprises CRISPR RNA (crRNA) and tracrRNA, wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target;
ii. a Cas molecule, wherein said Cas molecule cleaves nucleic acid when the crRNA is 100% complementary to the target nucleic acid with the given region, but does not cleave nucleic acid, or cleaves nucleic acid less efficiently, when the crRNA is hybridized with a variant which comprises one or two mismatches in the given region; and
iii. a nucleic acid probe, wherein said nucleic acid probe is cleavable by the Cas molecule, and further wherein the probe gives a different signal when cleaved compared to when it is not cleaved; and
b. a detection means, whereby said detection means indicates whether a target nucleic acid or variant thereof is present.

29-37. (canceled)

38. The method of claim 28, wherein the target and/or variant thereof comprises secondary structure, wherein said secondary structure affects crRNA hybridization kinetics.

39-51. (canceled)

52. A method for optimizing discrimination between a target nucleic acid and at least one variant of the target nucleic acid during detection, the method comprising:
a. determining a target nucleic acid and a variant of the target nucleic acid for detection, wherein discrimination between the target and the variant is desired;
b. designing an sgRNA molecule, wherein said sgRNA comprises crRNA and tracrRNA, wherein said crRNA hybridizes with the target nucleic acid when the crRNA is 100% complementary to the target nucleic acid with a given region, and wherein the crRNA also hybridizes with a variant of the target nucleic acid, wherein said variant comprises one or two mismatches within a given region of the target;
c. optimizing discrimination between the target nucleic acid and the variant thereof by manipulating design of the gRNA molecule, wherein said manipulation is done by:
i. determining which sequence or secondary structure of the sgRNA yields optimum discrimination between the target nucleic acid and a variant thereof;
ii. designing an sgRNA molecule accordingly.

53-71. (canceled)
